(19) United States
(12) Patent Application Publication
(10) Pub. No.: US 2003/0199424 A1
(43) Pub. Date: Oct. 23, 2003

(54) Method of treatment and/or prophylaxis

(76) Inventors: Maree Therese Smith, Bardon (AU); Lindsay Charles Brown, Sinnamon Park (AU)

Correspondence Address: MINTZ, LEVIN, COHN, FERRIS, GLOVSKY, AND POPEO, P.C., ONE FINANCIAL CENTER, BOSTON, MA, 02111, US

(21) Appl. No.: 10/393,056

(22) Filed: Mar. 20, 2003

Related U.S. Application Data
(60) Provisional application No. 60/365,858, filed on Mar. 20, 2002.

(51) Int. Cl. A61K 31/00; A61K 31/7076; A61K 31/7072

(52) U.S. Cl. 514/1; 514/45; 514/49; 514/283; 514/386

(57) ABSTRACT

The present invention is directed to the use of angiotensin II receptor I (AT₁ receptor) antagonists for the treatment, prophylaxis, reversal and/or symptomatic relief of a neuropathic condition, especially a peripheral neuropathic condition such as painful diabetic neuropathy, in vertebrate animals and particularly in human subjects. The present invention also discloses the use of AT₁ receptor antagonists for preventing, attenuating or reversing the development of reduced opioid sensitivity, and more particularly reduced opioid analgesic sensitivity, in individuals and especially in individuals having, or at risk of developing, a neuropathic condition.

### RELATED APPLICATION

This application claims the benefit of U.S. Provisional Application No. 60/365,858 filed Mar. 20, 2002, and which is hereby incorporated herein in its entirety by reference.

### FIELD OF THE INVENTION

THIS INVENTION relates generally to compounds that are useful in the prevention and amelioration of signs and symptoms associated with a neuropathic condition. More particularly, the present invention relates to the use of angiotensin II receptor I (AT₁ receptor) antagonists for the treatment, prophylaxis, reversal and/or symptomatic relief of a neuropathic condition, especially a peripheral neuropathic condition such as painful diabetic neuropathy, in vertebrate animals and particularly in human subjects. The AT₁ receptor antagonists may be provided alone or in combination with other compounds such as those that are useful in the control of neuropathic conditions. The present invention also extends to the use of AT₁ receptor antagonists for preventing, attenuating or reversing the development of reduced opioid sensitivity and more particularly reduced opioid analgesic sensitivity. In a preferred embodiment, the present invention encompasses the use of AT₁ receptor antagonists for preventing, attenuating or reversing the development of reduced analgesic sensitivity to an opioid receptor agonist in an individual afflicted with, or at risk of developing, a neuropathic condition.

### BACKGROUND OF THE INVENTION

Symmetric sensory polyneuropathy (usually called diabetic neuropathy) is the most common form of peripheral neuropathy in the western world with a prevalence of 7% within a year of diagnosis of diabetes, 50% for patients with diabetes for more than 25 years and 100% if subclinical, non-symptomatic neuropathy is included (Sima and Sugimoto, 1999, Diabetologia, 42: 773-788). Although epidemiological studies such as the diabetes control and complications trial (DCCT-Research group, 1995, Ann Intern Med 122: 561-568) show that aggressive blood glucose control can reduce the development of diabetic neuropathy by as much as 60% (DCCT-Research group, 1995, supra), tight glycaemic control is extremely difficult for many diabetic patients to achieve. Moreover, there are large numbers of patients (300,000 estimated in Australia [International Diabetes Institute website. www.diabetes.com.au Accessed Feb. 19, 2002] and 5 million in the USA [American Diabetes Association website. www.diabetes.org Accessed Feb. 19, 2002]) with undiagnosed type 2 diabetes who unknowingly have markedly elevated blood glucose concentrations for prolonged periods, and hence are at high risk of developing this longterm complication of diabetes.

Apart from tight glycaemic control, there are no currently available treatments that are known to prevent/attenuate or reverse the development of painful diabetic neuropathy (PDN) in patients. Hence clinical guidelines for the management of diabetic patients emphasise the importance of tight glycaemic control, for the prevention of the development of the longterm microvascular complications of diabetes, including PDN.

Furthermore, there are no treatments that can prevent or reverse the development of PDN and hence the available medications for its treatment are essentially palliative i.e. targeted to providing symptomatic relief.

Specifically, PDN is a debilitating long-term neurological complication of diabetes mellitus associated with sensory dysfunction of the peripheral nerves. Patients with PDN typically report unrelenting chronic pain primarily localised to the lower limbs, marked by burning and tingling sensations combined with deep muscular aches (Fox et al., 1999, Pain, 81: 307-316.). The symptomatic phase of PDN is often acute in onset and may persist for many years (Thomas and Scadding, 1987, Treatment of pain in diabetic neuropathy. In: P. J. Dyck, et al., (Eds.), Diabetic Neuropathy, 1987 pp. 216-222) before being paradoxically replaced by a complete loss of sensory function, reflecting overall peripheral nerve degeneration (Malik, 1997, Diabetes, 46(Suppl 2): S50-S53). Clinically, PDN is of particular concern as it is associated with poor patient outcomes for currently available analgesic agents. Consequently, improved alternative pharmacological interventions are required.

Although PDN is primarily attributed to hyperglycaemia, its exact pathogenesis remains to be defined (Stevens, 1995, Diabet Med, 12: 292-295; Feldman and Windebank, Growth Factors and Periperal Neuropathy. In: P. J. Dycke and P. K. Thomas (Eds), W. B. Saunders Company, Philadelphia, p. 575; Arezzo, 1999, Am. J. Med., 107: 9S-16S). It is clear that the pathobiochemistry of this condition is highly complex, involving an array of metabolic and vascular factors operating in concurrent and interdependent relationships. For ease of explanation, the aetiology of diabetic neuropathy is often categorized under one of two headings, namely ‘metabolic’ or ‘vascular’ (Cameron et al., 1993, Diabetologia, 30: 46-48). However, controversy arises regarding the relative contribution of metabolic and vascular abnormalities that underlie the development of neuropathy and is the subject of much current research (Greene et al., 1990, Annu. Rev. Med., 41: 303-317).

Multiple authors have proposed that metabolic changes such as excessive activity of the polyol pathway (Dvomik, 1992, J. Diabetes Complications 6: 25-34), altered myo-inositol and phosphoinositide metabolism (Greene et al., 1988, Diabetes Metab. Rev. 4: 201-221), impaired essential fatty acid metabolism (Horrobin, 1988, Prostaglandins Leukot. Essent. Fatty Acids 31: 181-197), the formation of advanced glycation end-products (Brownlee et al., 1988, N. Eng. J. Med 318: 1315-1321; Baynes, 1991, Diabetes 40: 405-412), and oxidative stress (Baynes, 1991, supra), may induce the microvascular complications of diabetes (Cameron et al., 1993, supra). At present however, it is unclear whether the metabolic or vascular abnormalities (or both) are associated with these adverse changes in peripheral nerves (Feldman and Windebank, 1999, supra). Additionally, Feldman and Windebank have pointed out that secondary dysfunction of components of the peripheral nervous system, including the perineurium or extracellular matrix, may underlie the development of diabetic neuropathy (Feldman and Windebank, 1999, supra). Overall, there is general agreement that there are multiple metabolic abnormalities underlying the development of diabetic neuropathy, whereby there are inter-related deviations of individual metabolic pathways that are mutually perpetuating (Sima and Sugimoto, 1999, supra). For example, enhanced advanced glycation end products (AGE) formation and activation of the polyol pathway may lead to oxidative stress; oxidative stress may accelerate AGE formation and lead to both activation of protein kinase C and altered growth factor expression, and so on (Baynes and Thorpe, 1999, Diabetes 48: 1-9).

It has also been proposed that these metabolic derangements are translated into neuropathic nerve injury primarily by their actions on nerve vasculature resulting in decreased perineurial blood flow and endoneurial hypoxia (Cameron et al., 1993, supra). On this basis, treatments which could potentially improve perineurial blood flow could have therapeutic benefit (Cameron et al., 1993, supra) for the treatment of PDN (Malik, 2000, Ann Med, 32: 1-5).

Microvascular disease is the hallmark of other long-term diabetic complications particularly retinopathy and nephropathy (Haak et al. 1998, Exp Clin Endocrinol Diabetes, 106: 45-50; Calles-Escandon and Cippola, 2001, Endocr Rev, 22: 36-52). Similarly, PDN in humans has been associated with advanced microangiopathy localised to the endoneurial capillaries (Malik et al., 1993, Diabetologia, 36: 454-459), characterised by significantly increased basement membrane thickening and endothelial cell hyperplasia and hypertrophy culminating in lumenal occlusion (Dyck, et al. 1985, Proc Natl Acad Sci USA, 82: 2513-2517; Yasuda and Dyck, 1987, Neurology, 37: 20-28; Malik et al., 1989, Diabetologia, 32: 92-102). Indeed, the severity of these structural abnormalities has also been correlated with the clinical severity of PDN in human patients (Malik et al., 1989, supra; Malik et al., 1992, J Neurol Neurosurg Psychiatry, 55: 557-561; Giannini and Dyck, 1995, Ann Neurol, 37: 498-504) and are postulated to be preceded by microvascular dysfunction (Tooke, 1989, Br Med Bulletin, 45: 206-223) as indicated by increased capillary pressure and microvascular resistance in comparison with healthy individuals (Sandemann et al., 1992, N Eng J Med, 327: 760-764). However, the underlying mechanisms responsible for these functional and structural aberrations of the vasculature remain obscure, although metabolic insult to the vascular endothelium secondary to hyperglycaemia-induced oxidative stress has been suggested (Nishikawa et al., 2000, Kidney Int, 58 (Suppl 77): S26-S30; Soriano et al., 2001, Nat Med, 7:108-113).

Functionally, it is thought that these neurovascular abnormalities impair perfusion of the vasa nervorum producing a chronic hypoxic state within the peripheral nerve (Low et al., 1997, Diabetes, 46 (Suppl 2): S38-S42), thereby increasing oxidative stress and initiating secondary pathogenic processes including lipid peroxidation (Low et al., 1997, Diabetes, 46 (Suppl 2): S38-S42) and activation of protein kinase C (PKC) (Taher et al.,1993 Arch Biochem Biophys, 303: 260-266). Administration of vasodilatory agents to diabetic rodents however, can reverse deficits in nerve blood flow (NBF) and nerve conduction velocity (NCV) (thought to precede the development of PDN) without altering metabolic parameters (Yasuda et al., 1989, Diabetes, 38: 832-838; Cameron et al., 1991, Diabetologia, 40: 1652-1658).

Angiotensin-converting enzyme (ACE) is a member of the renin-angiotensin system involved in the regulation of blood pressure and is markedly enhanced in patients with diabetes (Van Dyk, et al., 1994, Eur J Clin Invest, 24: 463-467). The extent of this enhancement appears to be strongly correlated with the severity of other long-term microvascular complications of diabetes, viz nephropathy and retinopathy (Duntas et al., 1992, Diabetes Res Clin Pract, 16: 203-238). The resulting increased levels of the potent vasoconstrictor, angiotensin II (Ang II), have the potential to contribute to the perivascular hypoperfusion and nerve hypoxia reported in diabetic rodents and patients. Indeed, recent studies in diabetic rodents have suggested that Ang II antagonism may be a potentially important target for identification of novel therapeutic options for the treatment of PDN. Specifically, Kihara et al. (1999, Muscle Nerve, 22: 920-925), reported that the vasopressive potency of Ang II in the vasa nervorum of streptozotocin (STZ)-diabetic rats was augmented relative to that in control non-diabetic rats, consistent with reports of tissue specific increases in Ang II Type 1 (AT₁)-receptor density in diabetic rats relative to non-diabetic rats (Brown et al., 1997, J Endocrin, 154: 355-362). Ang II under certain circumstances can also induce endothelial damage owing to its mitogenic properties and regulatory influence on extracellular matrix proteins (Katz, 1990, J Mol Cell Cardiol, 22: 239-247). Taken together, this indirect evidence suggests that Ang II and possibly other members of the renin-angiotensin system may be intimately involved in attenuating endoneurial perfusion in the diabetic state and may, therefore, be attractive targets for the treatment and/or prevention of pain and/or pathology associated with PDN.

Several recent studies have explored the utility of renin-angiotensin system inhibitors, including ACE inhibitors and AT, receptor antagonists, for the prevention and/or attenuation of PDN. For example, an interventional study has found that the administration of ZD 8731 (an experimental AT₁-antagonist) to rats 4 wks after the induction of diabetes with streptozotocin (STZ) completely reverses the reduction in NCV to values not significantly different from those found in control non-diabetic rats (p>0.05) (Maxfield et al., 1993, Diabetologia, 12: 1230-1237). Additionally, there were significant improvements in the NBF deficits (p<0.05) which occurred independent of decreases in systemic blood pressure, thereby implicating Ang II as having a central role in inducing an increase in vasa nervorum resistance (Maxfield, 1993, supra). Other studies whereby ACE inhibitors have been given to block Ang II synthesis, have reported similar improvements in NBF and NCV deficits in STZ-diabetic rats (Cameron et al., 1993, supra; Kihara et al., 1999, supra) as well as improvements in NCV deficits in human patients with type I or type II diabetes (Malik, et al., 1998, Lancet, 352: 1978-1981). In all of these studies, NCV was used as the primary endpoint of neuropathy based on the art recognised view that NCV was objective, quantitative and reproducible as well as correlating with underlying nerve fibre abnormalities (Veves, et al., 1991, Diabet Med, 8: 917-921; Consensus report of the peripheral nerve society, 1995; Dyck, et al., 1997, Diabetes, 46 (Suppl 2): S5-S8). However, in all of the human trials investigating the efficacy of ACE inhibitors in PDN (Reja et al., 1995, Diab Med, 12: 307-309; Malik et al., 1998, supra), there was no improvement in either symptoms such as pain or neuropathic disability, despite quantifiable improvements in NCV. These findings also mirror the disappointing outcomes of clinical trials employing aldose reductase inhibitors (targeting the metabolic abnormality in peripheral nerves) whereby the aldose reductase inhibitors improved the so-called objective measures of NCV and NBF, but failed to improve symptoms of pain in diabetic patients (Pfeifer et al, 1997, Diabetes 46 Suppl 2: S82-9; Thomas, P. K., Mechanisms and Treatment of Pain. In: P. J. Dyck and P. K. Thomas (Eds.), Diabetic Neuropathy, W. B. Saunders Company, Philadelphia, 1999, pp. 387-397). Together, the results of these clinical studies have called into question the validity of the hypothesis that changes in both NBF and NCV are directly correlated with the development of PDN.

In fact, the perception that NCV-improving compounds, including the ACE inhibitors and AT, receptor antagonist of the above studies, could be useful in the prevention and/or attenuation of PDN has been further undermined by Malik et al. (2001, Acta Neuropathol (Berl) 101: 367-374) who showed unequivocally that neurophysiological (e.g., NCV) and neuropathological parameters do not discriminate between diabetic patients with painful and painless neuropathy.

Thus, in contrast to what was hypothesised previously, clinical trial evidence in diabetic patients indicates that ACE inhibitors are not useful for the treatment and/or alleviation of PDN. By analogy, other inhibitors of the renin-angiotensin system would also not be expected to be useful for the treatment and/or alleviation of PDN. Accordingly, there still remains a need for the provision of agents that are effective for treating and/or preventing the painful symptoms associated with this debilitating condition.

### SUMMARY OF THE INVENTION

The present invention discloses the discovery that AT, receptor antagonists such as candesartan are effective in the prevention and/or attenuation of the painful symptoms of PDN and of other neuropathies, including peripheral neuropathies. In one aspect, therefore, the invention provides methods for the treatment or prophylaxis of a neuropathic condition in a subject. In one embodiment, the neuropathic condition is treated or prevented by administering to the subject an effective amount of an AT₁ receptor antagonist. The AT₁ receptor antagonist is suitably administered in the form of a composition comprising a pharmaceutically acceptable carrier and/or diluent. The composition may be administered by injection, by topical application or by the oral route including sustained-release modes of administration, over a period of time and in amounts which are effective to treat and/or prevent the neuropathic condition. In one embodiment, the neuropathic condition is a peripheral neuropathic condition, especially painful diabetic neuropathy (PDN) or related condition. In another embodiment, the AT₁ receptor antagonist is candesartan or an analogue or derivative or prodrug thereof or a pharmaceutically compatible salt of these. In yet another embodiment, the patient is normotensive.

In accordance with the present invention, AT₁ receptor antagonists have been shown to prevent or attenuate the pain associated with a neuropathic condition. Thus, in another aspect, the invention provides methods for preventing or attenuating neuropathic pain, especially peripheral neuropathic pain, in a subject. In one embodiment, neuropathic pain is prevented or attenuated by administering to the subject an effective amount of an AT₁ receptor antagonist, which is suitably in the form of a composition comprising a pharmaceutically acceptable carrier and/or diluent.

The present invention also discloses the discovery that AT₁ receptor antagonists can act to prevent, attenuate or reverse the development of reduced opioid analgesic sensitivity. Thus, in yet another aspect, the invention provides methods for preventing, attenuating or reversing the development of reduced analgesic sensitivity to an opioid receptor agonist in a subject. In one embodiment, the development of this reduced analgesic sensitivity is prevented, attenuated or reversed by administering to the subject an effective amount of an AT₁ receptor antagonist. In one embodiment, the subject has, or is at risk of developing, a neuropathic condition, which is suitably a peripheral neuropathic condition such as PDN. The AT₁ receptor antagonist is suitably in the form of a composition comprising a pharmaceutically acceptable carrier and/or diluent. In another embodiment, the opioid receptor agonist is a μ-opioid receptor agonist or a compound which is metabolised or otherwise converted in vivo to a μ-opioid receptor agonist. For example, the μ-opioid receptor agonist may be selected from morphine, methadone, fentanyl, sufentanil, alfentanil, hydromorphone, oxymorphone, their analogues, derivatives or prodrugs and a pharmaceutically compatible salt of any one of these. Suitably, the opioid receptor agonist is morphine or an analogue or derivative or prodrug thereof, or a pharmaceutically compatible salt of these. In another embodiment the opioid receptor agonist is oxycodone or an analogue or derivative or prodrug thereof, or a pharmaceutically compatible salt of these.

The present invention also discloses the discovery that an AT₁ receptor antagonist may be administered together with an opioid analgesic, which agonises the same opioid receptor as an opioid receptor agonist that is the subject of reduced opioid analgesic sensitivity, for the production of analgesia in an individual. Thus, in yet another aspect, the present invention provides methods for producing analgesia in a subject having, or at risk of developing, reduced analgesic sensitivity to an opioid receptor agonist. In one embodiment, an AT, receptor antagonist is administered to the subject in an amount that is effective for preventing, attenuating or reversing the reduced opioid analgesic sensitivity. The AT₁ receptor antagonist is administered separately, simultaneously or sequentially with an opioid analgesic, which agonises the same opioid receptor as the opioid receptor agonist, in an amount that is effective for producing the analgesia. The AT₁ receptor antagonist and the opioid analgesic may be administered in the form of separate compositions each comprising a pharmaceutically acceptable carrier and/or diluent. In one embodiment, the AT, receptor antagonist and the opioid receptor agonist are administered together in the form of a single composition comprising a pharmaceutically acceptable carrier and/or diluent. In another embodiment, the subject has, or is at risk of developing, a neuropathic condition. The neuropathic condition is suitably a peripheral neuropathic condition such as PDN or related condition, which is associated with the development of reduced analgesic sensitivity to the opioid receptor agonist.

In still another aspect, the invention provides compositions for producing analgesia in a subject having, or at risk of developing, reduced analgesic sensitivity to an opioid receptor agonist. These compositions generally comprise an AT₁ receptor antagonist and an opioid analgesic, which agonises at least partially the same opioid receptor as the opioid receptor agonist, in effective amounts as broadly described above. In one embodiment, the subject exhibits, or is at risk of developing, a neuropathic condition, especially a peripheral neuropathic condition such as PDN or related condition.

In a further aspect, the present invention contemplates the use of an AT₁ receptor antagonist and of an opioid analgesic in the manufacture of a medicament for producing analgesia in a subject, especially in a subject who has, or is at risk of developing, a neuropathic condition, which is suitably a peripheral neuropathic condition such as PDN or related condition.

### BRIEF DESCRIPTION OF THE DRAWINGS

FIG. 1 is a graphical representation showing mean body weight of STZ-diabetic rats (i SEM) as a function of time with either high or low doses of candesartan for 24 wks following STZ administration. (n=11-27 for high dose candesartan (2.0 mg/kg/day), n=6-7 for low dose candesartan (0.5 mg/kg/day) and n=5-6 for control STZ-diabetic rats).

FIG. 2 is a graphical representation showing the effects of candesartan on the temporal changes in the mean (±SEM) paw withdrawal thresholds (g) in STZ-diabetic rodents assessed using Von Frey filaments. (n=11-27 for high-dose candesartan (2.0 mg/kg/day), n=6-7 for low-dose candesartan (0.5 mg/day/kg) and n=5-6 for control STZ-diabetic rats). The dashed line indicates the mean (±SEM) range of values for paw withdrawal thresholds for non-diabetic control rats. For STZ-diabetic rats that received the once daily low-dose oral candesartan prevention dosing protocol, paw withdrawal latencies were significantly lower than the corresponding values determined in rats that received high-dose candesartan by 22 wks post-STZ. (**** p<0.0001).

FIG. 3 is a graphical representation showing the effects of morphine on the temporal change in the mean (±SEM) degree of antinociception (expressed as the % maximum possible effect, %MPE) versus time curve following subcutaneous (s.c.) bolus dose administration of morphine in control STZ-diabetic rats. At 3 and 9 wks post-STZ, STZ-diabetic rats received the ED₅₀ s.c. morphine dose (6.1 mg/kg, n=6). The morphine dose was increased to 14 and 18 mg/kg at 12 (n=6) and 24 (n=5) wks post-STZ respectively.

FIG. 4 is a graphical representation showing a 3-fold decrease in potency of oxycodone at 24 wks post-STZ administration. In particular, the mean (±SEM) degree of antinociception (%MPE) versus time curve is shown following bolus s.c. administration of oxycodone in control STZ-diabetic rats. Dose ranging experiments were conducted to determine the approximate ED₅₀ oxycodone doses at 3 (n=6), 9 (n=6), 12 (n=6) and 24 (n=5) wks post-STZ.

FIG. 5 is a graphical representation showing that chronic once-daily oral administration of an anti-hypertensive dose of candesartan (2.0 mg/kg/day) to STZ-diabetic rats preserved the antinociceptive potency of morphine for the full 24 wk duration of the study. Specifically, this figure shows a dose-dependent increase in the mean (±SEM) degree of antinociception (%MPE) evoked by s.c. bolus doses of morphine given to 3 wks post-STZ diabetic rats that received chronic once daily anti-hypertensive doses of oral candesartan (2.0 mg/kg/day). The morphine doses administered were 0.8 mg/kg (n=6), 2.4 mg/kg (n=6) and 6.0 mg/kg (n=6), consistent with the doses of s.c. morphine used to produce a dose-response curve for morphine in non-diabetic control rats previously in our laboratory (Saini, K., 2000, “Differential potency of single-doses of subcutaneous morphine and oxycodone for the relief of mechanical allodynia in Dark Agouti rats with CCI and STZ-diabetic neuropathic pain.” On-Course Hons Research Article, School of Pharmacy, The University of Queensland).

FIG. 6 is a graphical representation showing that once-daily oral administration of candesartan at an anti-hypertensive dose (2 mg/kg/day) completely prevented the temporal loss of morphine potency and efficacy throughout the 24 wk post-STZ study period relative to non-diabetic control rat. In particular, the mean (±SEM) dose-response curves for s.c. morphine in STZ-diabetic rats, chronically administered once daily anti-hypertensive doses of oral candesartan (2.0 mg/kg/day) determined at 3 (n=18), 9 (n=18), 12 (n=22) and 24 (n=21) wks post-STZ, did not differ significantly from the dose-response curve for s.c. morphine in control non-diabetic rats (n=18). Dose-response curves were generated using non-linear regression as implemented in GraphPad Prism™. The corresponding mean (±SEM) ED₅₀ values for candesartan-treated STZ-diabetic rats at 3, 9, 12 and 24 wks and control untreated non-diabetic rats were 2.5 (±0.5) mg/kg, 2.3 (±0.4) mg/kg, 2.1 (+0.3) mg/kg, 2.4 (±0.4) mg/kg and 2.9 (+0.3) mg/kg respectively.

FIG. 7 is a graphical representation showing that the morphine dose-response curve in control non-diabetic rats that received chronic once-daily high-dose oral candesartan treatment (2.0 mg/kg/day) was not significantly different from that for non-diabetic control rats that did not receive oral candesartan treatment. Specifically, this graph shows the dose-response curves (mean±SEM) for s.c. morphine in control non-diabetic rats administered high-dose candesartan (n=18) and in weight-matched non-diabetic protocol controls (n=18) that received once daily oral vehicle (DMSO:water, 10:90) in comparison to untreated control non-diabetic rats. Dose-response curves were generated by using non-linear regression as implemented in GraphPad Prism™. ED₅₀ values for control high-dose oral candesartan-treated non-diabetic rats and untreated control non-diabetic rats were 2.3±0.3 mg/kg and 2.9±0.3 mg/kg respectively.

FIG. 8 is a graphical representation showing that the increase in the time to reach peak morphine antinociception following bolus doses of s.c. morphine in high-dose oral candesartan-treated STZ-diabetic rats occurred independent of candesartan treatment. In particular, for rats treated with chronic once-daily oral candesartan (2.0 mg/kg/day), the mean (+SEM) area under the degree of antinociception (%MPE) versus time curve evoked by s.c. bolus doses of morphine (2.4 mg/kg) at 24 wks post-STZ administration (135±9.8%MPE.h.) did not differ significantly from that found in weight-matched control non-diabetic rats (149±18.8%MPE.h). However, the mean (±SEM) time to achieve the peak antinociceptive effect of s.c. morphine was significantly delayed (p<0.05) in STZ-diabetic rats (60 min) (n=8) when compared with control non-diabetic rats (45 min) (n=6), regardless of candesartan treatment.

FIG. 9 is a graphical representation showing that the potency of oxycodone in STZ-diabetic rats was preserved by once-daily oral administration of an anti-hypertensive dose of candesartan (2.0 mg/kg/day) with no significant alterations in the timing for peak antinociceptive effect during the 24 wk experimental period. Specifically, this graph shows the dose-dependent increase in the mean (±SEM) degree of antinociception (%MPE) evoked by s.c. bolus doses of oxycodone in 3 wks post-STZ diabetic rats that received chronic once daily anti-hypertensive doses of oral candesartan (2.0 mg/kg/day). The oxycodone doses administered were 0.9 mg/kg (n=6), 1.2 mg/kg (n=6) and 2.2 mg/kg (n=6), consistent with the doses of s.c. oxycodone used to produce the dose-response curve for oxycodone in non-diabetic control rats, previously in our laboratory (Saini, 2000).

FIG. 10 is a graphical representation showing that once-daily oral administration of candesartan at an anti-hypertensive dose (2 mg/kg/day) completely prevented the temporal loss of oxycodone potency and efficacy throughout the 24 wk post-STZ study period relative to non-diabetic control rats. In particular, the graph shows the mean (±SEM) dose-response curves for s.c. oxycodone in STZ-diabetic rats chronically administered once daily anti-hypertensive doses of oral candesartan (2.0 mg/kg/day) determined at 3 (n=18), 9 (n=18), and 24 (n=18) wks post-STZ did not differ significantly for the dose-response curve for s.c. oxycodone determined in control untreated non-diabetic rats. Dose-response curves were generated by using non-linear regression as implemented in GraphPad Prism™. The corresponding mean (±SEM) ED₅₀ values for candesartan-treated STZ-diabetic rats at 3, 9 and 24 wks post-STZ and control untreated non-diabetic rats were 1.4 (±0.1) mg/kg, 1.3 (±0.1) mg/kg, 1.1 (±0.1) mg/kg and 1.2 (±0.1) mg/kg respectively.

FIG. 11 is a graphical representation showing that the protective effect of high-dose candesartan on oxycodone potency in STZ-diabetic rats appeared to occur independent of direct alterations by oral candesartan upon s.c. oxycodone administration. The graph shows the dose-response curves (mean±SEM) for s.c. oxycodone in control non-diabetic rats administered high-dose candesartan (n=18) and weight-matched non-diabetic protocol controls (n=18) that received once daily oral vehicle (DMSO:water, 10:90) in comparison to untreated control non-diabetic rats. Dose-response curves were generated by using non-linear regression as implemented in GraphPad Prism™. ED₅₀ values for candesartan-treated non-diabetic control rats and untreated non-diabetic control rats were 1.1 (±0.1) mg/kg and 1.2 (±0.1) mg/kg respectively.

FIG. 12 is a graphical representation showing that cessation of chronic high-dose oral candesartan treatment resulted in a decrease in the mean (±SEM) paw withdrawal threshold. The graph shows the mean (±SEM) paw withdrawal thresholds for 24 wks post-STZ diabetic rats (n=4-6) following cessation and subsequent re-initiation of chronic high-dose oral candesartan (2.0 mg/kg/day) administration. Cessation of chronic high-dose oral candesartan treatment for six wks resulted in a significant (p<0.0001) decrease in mean (±SEM) paw withdrawal thresholds in comparison to the values observed in the same rats immediately prior to candesartan cessation (wk 0). Re-initiation of once-daily high-dose oral candesartan (2.0 mg/kg/day) treatment for another six wks however, restored the paw withdrawal thresholds in these rats to values not significantly (p>0.05) different from those observed in the same rats immediately prior to cessation of candesartan treatment or non-diabetic control rats. * Death of one rat.

FIG. 13 is a graphical representation showing that cessation of once-daily high-dose oral candesartan treatment (2.0 mg/kg/day) resulted in a temporal loss of morphine potency and that re-initiation of once-daily oral candesartan (2.0 mg/kg/day) completely reversed this trend. The graph shows the mean (±SEM) degree of antinociception (%MPE) versus time curves following s.c. bolus dose administration of morphine (2.4 mg/kg) in the same STZ-diabetic rats during the “reversal protocol” pilot study.

FIG. 14 is a graphical representation showing that cessation of once-daily high-dose oral candesartan treatment (2.0 mg/kg/day) resulted in a small but insignificant decrease in the potency of s.c. oxycodone and that this decrease was completely reversed by re-initiation of once-daily oral candesartan (2.0 mg/kg/day). Specifically, this graph shows the mean (±SEM) degree of antinociception (%MPE) versus time curves following s.c. bolus administration of oxycodone (1.2 mg/kg) in the same STZ-diabetic rats during the reversal protocol pilot study.

FIG. 15 is a graphical representation showing that 4 wks of either once-daily candesartan or losartan administration by oral gavage, commencing at 12 wks post-STZ administration, preserved morphine's antinociceptive effects. Plot of the mean (±SEM) antinociceptive response (expressed as the percentage of the maximum possible response) evoked by single bolus doses of s.c. morphine (6.1 mg/kg) in STZ-diabetic adult male Dark Agouti rats. STZ-diabetic rats developed morphine hyposensitivity in a temporal manner such that all morphine efficacy was abolished by 16-wks post-STZ administration in control animals. By contrast, for STZ-diabetic rats that received once-daily oral treatment with either candesartan (2 mg/kg/day) or losartan (20 mg/kg/day), commencing at 12 wks post-STZ administration, morphine sensitivity was preserved. Specifically, for STZ-diabetic rats that received 4 wks treatment with once-daily oral candesartan, morphine sensitivity at 16-wks post-STZ did not differ significantly (p>0.05) from that determined in the same rats just prior to initiation of candesartan treatment. Similarly, for STZ-diabetic rats that received 4 wks treatment with once-daily oral losartan, morphine sensitivity at 16-wks post-STZ not significantly (p>0.05) different from that determined in the same rats prior to initiation of losartan treatment at 12 wks post-STZ.

### DETAILED DESCRIPTION OF THE INVENTION

1. Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e. to at least one) of the grammatical object of the article. By way of example, “an element” means one element or more than one element.

As used herein, the term “about” refers to a quantity, level, value, dimension, size, or amount that varies by as much as 30%, 20% or 10% to a reference quantity, level, value, dimension, size, or amount.

The term “allodynia” as used herein refers to the pain that results from a non-noxious stimulus i.e. pain due to a stimulus that does not normally provoke pain. Examples of allodynia include, but are not limited to, cold allodynia, tactile allodynia (pain due to light pressure or touch), and the like.

The term “analgesia” is used herein to describe states of reduced pain perception, including absence from pain sensations as well as states of reduced or absent sensitivity to noxious stimuli. Such states of reduced or absent pain perception are induced by the administration of a pain-controlling agent or agents and occur without loss of consciousness, as is commonly understood in the art. The term analgesia encompasses the term “antinociception”, which is used in the art as a quantitative measure of analgesia or reduced pain sensitivity in animal models.

The term “causalgia” as used herein refers to the burning pain, allodynia, and hyperpathia after a traumatic nerve lesion, often combined with vasomotor and sudomotor dysfunction and later tropic changes.

By “complex regional pain syndromes” is meant the pain that includes, but is not limited to, reflex sympathetic dystrophy, causalgia, sympathetically maintained pain, and the like.

Throughout this specification, unless the context requires otherwise, the words “comprise”, “comprises” and “comprising” will be understood to imply the inclusion of a stated step or element or group of steps or elements but not the exclusion of any other step or element or group of steps or elements.

By “effective amount”, in the context of treating or preventing a condition is meant the administration of that amount of active to an individual in need of such treatment or prophylaxis, either in a single dose or as part of a series, that is effective for the prevention of incurring a symptom, holding in check such symptoms, and/or treating existing symptoms, of that condition. The effective amount will vary depending upon the health and physical condition of the individual to be treated, the taxonomic group of individual to be treated, the formulation of the composition, the assessment of the medical situation, and other relevant factors. It is expected that the amount will fall in a relatively broad range that can be determined through routine trials.

By “hyperalgesia” is meant an increased response to a stimulus that is normally painful.

By “neuropathic pain” is meant any pain syndrome initiated or caused by a primary lesion or dysfunction in the peripheral or central nervous system. Examples of neuropathic pain include, but are not limited to, thermal or mechanical hyperalgesia, thermal or mechanical allodynia, diabetic pain, entrapment pain, and the like.

“Nociceptive pain” refers to the normal, acute pain sensation evoked by activation of nociceptors located in non-damaged skin, viscera and other organs in the absence of sensitization.

The term “opioid receptor agonist” as used herein refers to any compound, which is optionally in the form of a pharmaceutically compatible salt, and which upon administration is capable of binding to an opioid receptor and causing agonism, partial agonism or mixed agonism/antagonism of the receptor. Metabolites of administered compounds are also encompassed by the term opioid receptor agonists. Preferred opioid receptor agonists are those that produce analgesia.

The term “pain” as used herein is given its broadest sense and includes an unpleasant sensory and emotional experience associated with actual or potential tissue damage, or described in terms of such damage and includes the more or less localised sensation of discomfort, distress, or agony, resulting from the stimulation of specialised nerve endings. There are many types of pain, including, but not limited to, lightning pains, phantom pains, shooting pains, acute pain, inflammatory pain, neuropathic pain, complex regional pain, neuralgia, neuropathy, and the like (Dorland's Illustrated Medical Dictionary, 28^{th} Edition, W. B. Saunders Company, Philadelphia, Pa.). The goal of treatment of pain is to reduce the degree of severity of pain perceived by a treatment subject.

By “pharmaceutically acceptable carrier” is meant a solid or liquid filler, diluent or encapsulating substance that may be safely used in topical, local or systemic administration.

The term “pharmaceutically compatible salt” as used herein refers to a salt which is toxicologically safe for human and animal administration. This salt may be selected from a group including hydrochlorides, hydrobromides, hydroiodides, sulphates, bisulphates, nitrates, citrates, tartrates, bitartrates, phosphates, malates, maleates, napsylates, fumarates, succinates, acetates, terephthalates, pamoates and pectinates.

The term “prodrug” is used in its broadest sense and encompasses those compounds that are converted in vivo to an AT₁ receptor antagonist or to an opioid receptor agonist according to the invention. Such compounds would readily occur to those of skill in the art, and include, for example, compounds where a free hydroxy group is converted into an ester derivative.

The terms “reduced opioid analgesic sensitivity”, “reduced analgesic sensitivity to an opioid receptor agonist” and the like are used interchangeably herein to refer to an abrogated, impaired or otherwise reduced analgesia produced by the administration of an amount or concentration of an opioid receptor agonist, which would otherwise produce analgesia in an opioid-naive individual, especially in an opioid-naive individual who does not have a neuropathic pain condition, more especially in an opioid-naive individual who does not have a peripheral neuropathic pain condition and even more especially in an opioid-naive non-diabetic individual.

The terms “subject” or “individual” or “patient”, used interchangeably herein, refer to any subject, particularly a vertebrate subject, and even more particularly a mammalian subject, for whom therapy or prophylaxis is desired. Suitable vertebrate animals that fall within the scope of the invention include, but are not restricted to, primates, avians, livestock animals (e.g., sheep, cows, horses, donkeys, pigs), laboratory test animals (e.g., rabbits, mice, rats, guinea pigs, hamsters), companion animals (e.g., cats, dogs) and captive wild animals (e.g., foxes, deer, dingoes). A preferred subject is a human in need of treatment or prophylaxis for a peripheral neuropathic condition, especially PDN or related condition. However, it will be understood that the aforementioned terms do not imply that symptoms are present.

2. Methods for the Treatment and/or Prophylaxis of Peripheral Neuropathic Conditions

The present invention arises from the unexpected discovery that, in contrast to ACE inhibitors, AT₁ receptor antagonists such as candesartan are effective in the prevention and/or attenuation of the painful symptoms of PDN and of other neuropathies, including peripheral neuropathies. Additionally, the AT₁ receptor antagonists, prevented, attenuated and/or reversed the development of hyposensitivity, and more particularly analgesic hyposensitivity, to an opioid receptor agonist (e.g. morphine or oxycodone) in a dose-dependent fashion. These discoveries are based on pre-clinical data which show that candesartan administration to STZ-diabetic rats causes a dose-dependent attenuation in the (i) onset and development of tactile allodynia, the defining symptom of PDN; and (ii) the loss of morphine or oxycodone sensitivity for the alleviation of tactile allodynia. The present inventors have also found unexpectedly that candesartan is efficacious for the reversal of established PDN in STZ-diabetic rats. Remarkably, such desirable outcomes occurred without alterations to metabolic parameters, including persistently elevated blood glucose concentrations, indicating that the beneficial effects of candesartan were obtained in the presence of profound hyperglycaemia, a condition normally associated with the development of PDN.

Accordingly, the present invention provides methods for treating and/or preventing neuropathic conditions, wherein the methods generally comprise administering to an individual afflicted with, or at risk of developing, a neuropathic condition, an effective amount of an AT₁ receptor antagonist, which is suitably in the form of a pharmaceutical composition. In accordance with the present invention, the AT₁ receptor antagonist can act to prevent or attenuate one or more symptoms associated with a neuropathic condition, which is suitably a peripheral neuropathic condition including, but not limited to, numbness, weakness, burning pain, and loss of reflexes. The pain may be severe and disabling. In a preferred embodiment, the symptom, which is the subject of the prevention and/or attenuation, is pain. Accordingly, in a related aspect, the invention provides methods for preventing and/or attenuating neuropathic pain, especially peripheral neuropathic pain, in an individual, comprising administering to the individual a pain-preventing or attenuating effective amount of an AT₁ receptor antagonist, which is suitably in the form of a pharmaceutical composition.

There are many possible causes of neuropathy and it will be understood that the present invention contemplates the treatment and/or prevention of any neuropathic condition regardless of the cause. In a preferred embodiment, the neuropathic conditions are a result of diseases of the nerves (primary neuropathy) and neuropathy that is caused by systemic disease (secondary neuropathy), such as but not limited to diabetic neuropathy, Herpes Zoster (shingles)-related neuropathy, uraemia-associated neuropathy, amyloidosis neuropathy, HIV sensory neuropathies, hereditary motor and sensory neuropathies (HMSN), hereditary sensory neuropathies (HSNs), hereditary sensory and autonomic neuropathies, hereditary neuropathies with ulcero-mutilation, nitrofurantoin neuropathy, tumaculous neuropathy, neuropathy caused by nutritional deficiency and neuropathy caused by kidney failure. Other causes include repetitive activities such as typing or working on an assembly line, medications known to cause peripheral neuropathy such as several AIDS drugs (DDC and DDI), antibiotics (metronidazole, an antibiotic used for Crohn's disease, isoniazid used for tuberculosis), gold compounds (used for rheumatoid arthritis), some chemotherapy drugs (such as vincristine and others) and many others. Chemical compounds are also known to cause peripheral neuropathy including alcohol, lead, arsenic, mercury and organophosphate pesticides. Some peripheral neuropathies are associated infectious processes (such as Guillian-Barre syndrome). In a preferred embodiment, the neuropathic condition is a peripheral neuropathic condition, which is suitably painful diabetic neuropathy (PDN) or related condition.

The neuropathic condition may be acute or chronic and, in this connection, it will be understood by persons of skill in the art that the time course of a neuropathy will vary, based on its underlying cause. With trauma, the onset of symptoms will be acute, or sudden, with the most severe symptoms at the onset. Inflammatory and some metabolic neuropathies have a subacute course extending over days to weeks. A chronic course over weeks to months usually indicates a toxic or metabolic neuropathy. A chronic, slowly progressive neuropathy over many years occurs with most hereditary neuropathies or with a condition termed chronic inflammatory demyelinating polyradiculoneuropathy (CIDP). Neuropathic conditions with symptoms that relapse and remit include the Guillian-Barre syndrome.

The AT₁ receptor antagonist includes and encompasses any active compound that binds to the AT₁ receptor subtype and that inhibits the effect of angiotensin II, including pharmaceutical compatible salts of the active compound. This category includes compounds having differing structural features. For example, in one embodiment, the AT, receptor antagonist is selected from the compounds listed in European Patent Application Publication No. 443983 (EP 443983), and especially in the compound claims of this publication. In a preferred embodiment of this type, the AT, receptor antagonist is (S)-N-(1-carboxy-2-methylprop-1-yl)-N-pentanoyl-N-[2′(1H-tetrazol-5-yl)biphenyl-4-ylmethyl]amine [valsartan] of the formula:

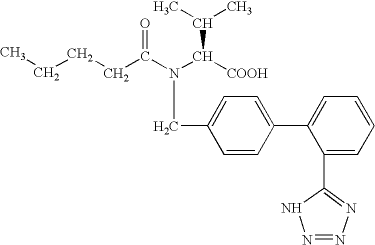

and its pharmaceutically compatible salts.

In another embodiment, the AT₁ receptor antagonist is selected from the compounds listed in European Patent Application Publication No. 253310 (EP 253310), and especially from the compounds listed in the claims of this publication. In a preferred embodiment of this type, the AT₁ receptor antagonist is the compound [losartan] of the following formula:

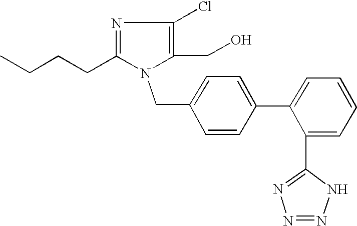

and its pharmaceutically compatible salts.

In yet another embodiment, the AT₁ receptor antagonist is selected from the compounds listed in European Patent Application Publication No. 403159 (EP 403159), and especially from the compounds listed in the claims of this publication. In a preferred embodiment of this type, the AT₁ receptor antagonist is the compound [eprosartan] of the following formula:

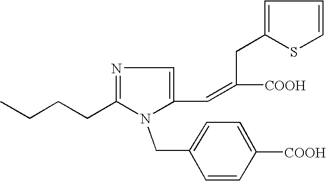

and its pharmaceutically compatible salts.

In still yet another embodiment, the AT₁ receptor antagonist is selected from the compounds listed in PCT Patent Application Publication No. WO 91/14679, and especially from the compounds listed in the claims of this publication. In a preferred embodiment of this type, the AT₁ receptor antagonist is the compound [irbesartan] of the following formula:

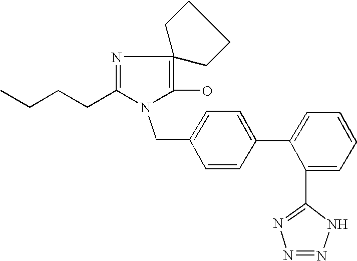

and its pharmaceutically compatible salts.

In a further embodiment, the AT₁ receptor antagonist is selected from the compounds listed in the European Patent Application Publication No. EP 420237 (EP 420237), and especially from the compounds listed in the claims of this publication. Preference is given in this regard to the compound [E-1477] of the following formula:

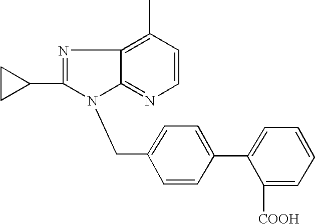

and its pharmaceutically compatible salts.

In yet a further embodiment, the AT₁ receptor antagonist is selected from the compounds listed in the European Patent Application Publication No. 502314 (EP 502314), and especially from the compounds listed in the claims of this publication. In a preferred embodiment of this type, the AT₁ receptor antagonist is the compound [telmisartan] of the following formula:

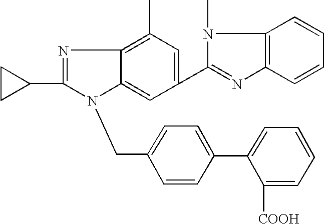

and its pharmaceutically compatible salts.

In still a further embodiment, the compounds listed in European Patent Application Publication No. 504888 (EP 504888), and especially those listed in the compound claims of this publication, can also be used as a basis for selecting the AT₁ receptor antagonist. In a preferred embodiment of this type, the AT₁ receptor antagonist is the compound [SC-52458] of the following formula:

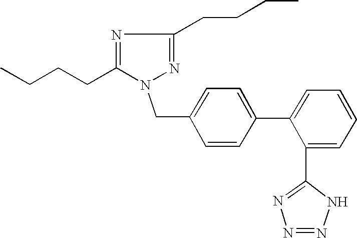

and its pharmaceutically compatible salts.

In even yet another embodiment, the compounds listed in European Patent Application Publication No. 514198 (EP 514198), and especially those listed in the compound claims of this publication, can also be used as a basis for selecting the AT₁ receptor antagonist. Preference is given in this regard to the compound [saprisartan] of the following formula:

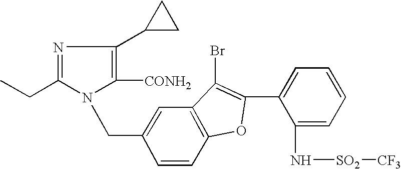

and its pharmaceutically compatible salts.

In another embodiment, the AT₁ receptor antagonist is selected from the compounds listed in the European Patent Application Publication No. 475206 (EP 475206), and especially from the compounds listed in the claims of this publication. In a preferred embodiment of this type, the AT₁ receptor antagonist is the compound [2-[N′-(2′-tetrazolylbiphenylmethyl)-N′(1-propyl)]amino-3-carboxy-pyridine] of the following formula:

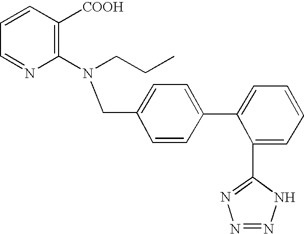

and its pharmaceutically compatible salts.

In even yet another embodiment, the AT₁ receptor antagonist is selected from the compounds listed in PCT Patent Application Publication No. WO 93/20816, and especially from the compounds listed in the claims of this publication. Preference is given in this regard to the compound [ZD-8731] of the following formula:

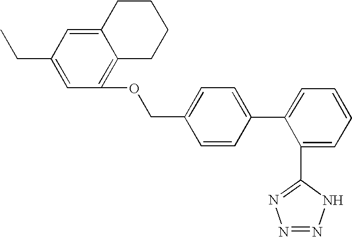

and its pharmaceutically compatible salts.

Suitably, the AT₁ receptor antagonist is selected from the compounds listed in the European Patent Application Publication No. 459136 (EP 459136), and especially from the compounds listed in the claims of this publication. In a preferred embodiment, the AT, receptor antagonist is the compound [candesartan] of the following formula:

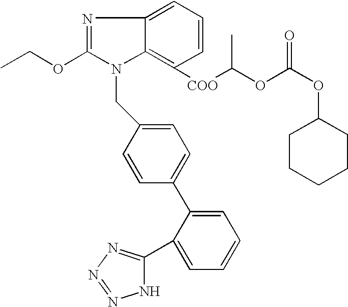

and its pharmaceutically compatible salts.

Alternatively, the AT₁ receptor antagonist may be selected from: 2,4-dimethyl-8-[[2′-(1H-tetrazol-5-yl)[1,1-biphenyl]-4-yl]methyl]-7H-pyrid o[2,3-d]pyrimidin-7-one [tasosartan] as for example described by Ellingboe et al. (1994, J Med Chem 37(4):542-50 and U.S. Pat. No. 5,149,699); 2-butyl-1-[2′-(1H-tetrazol-5-yl) -biphenyl-4-yl)methyl]-4-chloroimidazole-5-carboxylic acid [EXP-3174] as for example described by Nelson et al. (U.S. Pat. No. 5,663,186); 5-methyl-2-oxo-1,3-dioxolen-4-yl)methoxy-4-(1-hydroxy-1-methylethyl)-2-propyl-1-(4-[2-(tetrazol-5-yl)-phenyl]phenyl) methylimidazol-5-carboxylate [olmesartan, CS-866] as for example described by Mizuno et al., (1995 Eur J Pharmacol Oct 16;285(2):181-8); BMS-184698 as for example described by Smith, A. B. III et al. (1995, J. Org. Chem., 60:7837; and 3-(2′-(tetrazol-5yl)-1,1′-biphenyl-4-yl)methyl-5,7-dimethyl-2-ethyl-3H-imidazo[4,5-b]pyridine.

In an especially preferred aspect, the invention provides a method for treating and/or preventing a peripheral neuropathy in a subject, comprising administering to the subject a pharmaceutical composition comprising an effective amount of candesartan, or an analogue or derivative or prodrug thereof, or a pharmaceutically compatible salt of these, together with a pharmaceutically acceptable carrier and/or diluent.

An effective amount of an AT₁ receptor antagonist is one that is effective for the treatment or prevention of a neuropathic condition, including the prevention of incurring a symptom, holding in check such symptoms (e.g., pain), and/or treating existing symptoms associated with the neuropathic condition. Modes of administration, amounts of AT, receptor antagonist administered, and AT₁ receptor antagonist formulations, for use in the methods of the present invention, are discussed below. Whether the neuropathic condition has been treated is determined by measuring one or more diagnostic parameters indicative of the course of the disease, compared to a suitable control. In the case of an animal experiment, a “suitable control” is an animal not treated with the AT₁ receptor antagonist, or treated with the pharmaceutical composition without the AT₁ receptor antagonist. In the case of a human subject, a “suitable control” may be the individual before treatment, or may be a human (e.g., an age-matched or similar control) treated with a placebo. In accordance with the present invention, the treatment of pain includes and encompasses without limitation: (i) preventing pain experienced by a subject which may be predisposed to the condition but has not yet been diagnosed with the condition and, accordingly, the treatment constitutes prophylactic treatment for the pathologic condition; (ii) inhibiting pain initiation or a painful condition, i.e., arresting its development; (iii) relieving pain, i.e., causing regression of pain initiation or a painful condition; or (iv) relieving symptoms resulting from a disease or condition believed to cause pain, e.g., relieving the sensation of pain without addressing the underlying disease or condition.

The methods of the present invention are suitable for treating an individual who has been diagnosed with a neuropathic condition, who is suspected of having a neuropathic condition, who is known to be susceptible and who is considered likely to develop a neuropathic condition, or who is considered likely to develop a recurrence of a previously treated neuropathic condition. Where the individual to be treated is normotensive, the AT₁ receptor antagonist will suitably be administered in amounts below that required to cause a reduction in blood pressure. Where the individual to be treated is hypertensive, the AT₁ receptor antagonist will suitably be used in amounts usually employed to treat hypertension.

The present invention further provides a method for preventing, attenuating and/or reversing the development of reduced analgesic sensitivity to an opioid receptor agonist in a subject afflicted with, or at risk of developing, a neuropathic condition, comprising administering to the subject an effective amount of an AT₁ receptor antagonist and optionally a pharmaceutically acceptable carrier and/or diluent. The opioid receptor agonist that is the subject of the reduced analgesic sensitivity is suitably a μ-opioid receptor agonist or a compound that is metabolised or otherwise converted in vivo to a μ-opioid receptor agonist. For example, the μ-opioid receptor agonist may be selected from morphine, methadone, fentanyl, sufentanil, alfentanil, hydromorphone, oxymorphone, their analogues, derivatives or prodrugs and pharmaceutically compatible salts of these. In an especially preferred embodiment, the μ-opioid receptor agonist is morphine or an analogue or derivative or prodrug thereof or a pharmaceutically compatible salt of these. In another embodiment the opioid analgesic is a κ₂-opioid receptor agonist, which is suitably metabolised or otherwise converted in vivo to a μ-opioid receptor agonist. The κ₂-opioid receptor agonist is suitably any compound which upon administration is capable of binding to a κ₂-opioid receptor and causing agonism, partial agonism or mixed agonism/antagonism of that receptor, and whose antinociceptive effects are attenuated or otherwise impaired by nor-BNI (nor-binaltorphimine; a putatively selective κ₁/κ₂-opioid receptor ligand) and which does not displace the binding of the κ₁-selective radioligand, [³H]U69,593, from rat brain membranes. Metabolites of administered compounds are also encompassed by the term opioid receptor agonists. In a preferred embodiment of this type, the K₂-opioid receptor agonist is oxycodone or an analogue or derivative or prodrug thereof or a pharmaceutically compatible salt of these.

In accordance with the present invention, it is proposed that AT₁ receptor antagonists can prevent, attenuate and/or reverse the development of reduced analgesic sensitivity to an opioid receptor agonist and thus capacitate the opioid receptor agonist to provide pain relief. The reduced analgesic sensitivity may relate to the development of tolerance to an opioid receptor agonist, which results from the chronic administration of that agonist or to the development of opioid receptor agonist hyposensitivity associated with a neuropathic condition. Accordingly, in another aspect, the present invention provides a method for producing analgesia in a subject who exhibits, or is at risk of developing, reduced analgesic sensitivity to an opioid receptor agonist. These methods generally comprise administering separately, simultaneously or sequentially to the subject an AT₁ receptor antagonist and an opioid analgesic, which agonises the same opioid receptor as the opioid receptor agonist that is the subject of the reduced analgesic sensitivity, wherein the AT₁ receptor antagonist is administered in an amount that is effective for preventing, attenuating and/or reversing the reduced analgesic sensitivity to the opioid receptor agenist, and wherein the opioid analgesic is administered in an amount that is effective for producing the analgesia, which has been capacitated or otherwise rendered possible by the administration of the AT₁ receptor antagonist. The AT₁ receptor antagonist and the opioid analgesic, are suitably in association with a pharmaceutically acceptable carrier and/or diluent, and may be administered separately or in combination with each other.

In a preferred embodiment, the AT₁ receptor antagonist and the opioid analgesic are administered together for the treatment and/or prophylaxis of the painful symptoms associated with a neuropathic condition, which is suitably a peripheral neuropathic condition such as PDN or a related condition. The AT₁ receptor antagonist and the opioid analgesic, which are suitably in association with a pharmaceutically acceptable carrier and/or diluent, may be administered separately or in combination with each other or, in certain embodiments, with compositions having other useful anti-neuropathic properties or compounds which otherwise facilitate amelioration of the symptoms and signs of the neuropathic condition of interest.

Not wishing to be bound by any one particular theory or mode of operation, it is proposed that AT₁ receptor antagonists induce a direct or indirect physiological effect on opioid receptors to render them capable of being activated by their cognate opioid-receptor agonists, thereby producing pain relief. Thus, in another embodiment, the invention provides methods for producing analgesia in a subject who exhibits, or is at risk of developing, a condition associated with opioid analgesic hyposensitivity, wherein the methods generally comprise administering separately, simultaneously or sequentially to the subject an AT₁ receptor antagonist in an amount that is effective for rendering the opioid receptor capable of being activated by a cognate opioid receptor agonist, together with said cognate opioid receptor agonist in an amount that is effective for activating said opioid receptor and producing analgesia in the subject.

3. Compositions

Another aspect of the present invention provides compositions for treating, preventing and/or relieving the symptoms of a neuropathic condition, comprising an effective amount of an AT₁ receptor antagonist and a pharmaceutically acceptable carrier and/or diluent.

In yet another aspect, the invention provides compositions for producing analgesia and especially for treating, preventing and/or alleviating the painful symptoms of a neuropathic condition. These compositions generally comprise an AT, receptor antagonist which is present in an amount that is effective for preventing, attenuating or reversing the development of reduced analgesic sensitivity to an opioid receptor agonist as well as an opioid analgesic, which agonises at least partially the same opioid receptor as the opioid receptor agonist, and which is present in an amount that is effective for producing analgesia or for treating and/or preventing the painful symptoms of the neuropathic condition.

Any known AT₁ receptor antagonist and/or opioid analgesic compositions can be used in the methods of the present invention, provided that the AT₁ receptor antagonist and/or opioid analgesic are pharmaceutically active. A “pharmaceutically active” AT₁ receptor antagonist is in a form which results in the treatment and/or prevention of a neuropathic condition, including the prevention of incurring a symptom, holding in check such symptoms, and/or treating existing symptoms associated with the neuropathic condition, when administered to an individual. A “pharmaceutically active” AT₁ receptor antagonist also includes within its scope a form of AT₁ receptor antagonist which results in preventing or attenuating reduced opioid sensitivity or the development of hyposensitivity to an opioid receptor agonist. A “pharmaceutically active” opioid analgesic is in a form which activates, or which has been rendered capable of activating, or is metabolised or converted in vivo to be capable of activating, the corresponding opioid receptor.

The effect of compositions of the present invention may be examined by using one or more of the published models of pain/nociception or of neuropathy, especially peripheral neuropathy, and more especially PDN, known in the art. This may be demonstrated, for example using a model which assesses the onset and development of tactile allodynia, the defining symptom of PDN, as for example described herein. The analgesic activity of the compounds of this invention can be evaluated by any method known in the art. Examples of such methods are the Tail-flick test (D'Amour et al. 1941, J. Pharnacol. Exp. and Ther. 72: 74-79); the Rat Tail Immersion Model, the Carrageenan-induced Paw Hyperalgesia Model, the Formalin Behavioral Response Model (Dubuisson et al., 1977, Pain 4: 161-174), the Von Frey Filament Test (Kim et al., 1992, Pain 50: 355-363), the Radiant Heat Model, the Cold Allodynia Model (Gogas et al., 1997, Analgesia 3: 111-118), the paw pressure test (Randall and Selitto, 1957, Arch Int Pharmacodyn 111: 409-419) and the paw thermal test (Hargreaves et al., 1998, Pain 32: 77-88). An in vivo assay for measuring the effect of a test compound on the tactile allodynia response in neuropathic rats is described in Example 2. Compositions which test positive in such assays are particularly useful for the prevention, reduction, or reversal of pain in a variety of pain-associated conditions or pathologies including cancer, and are especially useful for the prevention, reduction, or reversal of neuropathic pain found, for example, in diabetic patients.

The active compounds of the present invention may be provided as salts with pharmaceutically compatible counterions. Pharmaceutically compatible salts may be formed with many acids, including but not limited to hydrochloric, sulfuric, acetic, lactic, tartaric, malic, succinic, etc. Salts tend to be more soluble in aqueous or other protonic solvents that are the corresponding free base forms.

Pharmaceutical compositions suitable for use in the present invention include compositions wherein the pharmaceutically active compounds are contained in an effective amount to achieve their intended purpose. The dose of active compounds administered to a patient should be sufficient to achieve a beneficial response in the patient over time such as a reduction in at least one symptom associated with a neuropathic condition, which is suitably neuropathic pain such as diabetic neuropathic pain. The quantity of the pharmaceutically active compounds(s) to be administered may depend on the subject to be treated inclusive of the age, sex, weight and general health condition thereof. In this regard, precise amounts of the active compound(s) for administration will depend on the judgement of the practitioner. In determining the effective amount of the active compound(s) to be administered in the treatment or prophylaxis of the neuropathic condition, the physician may evaluate numbness, weakness, pain, and loss of reflexes. In any event, those of skill in the art may readily determine suitable dosages of the AT₁ receptor antagonists and/or opioid receptor agonists of the invention.

In one embodiment, and dependent on the intended mode of administration, the AT₁ receptor antagonist-containing compositions will generally contain about 0.1% to 90%, about 0.5% to 50%, or about 1% to about 25%, by weight of AT₁ receptor antagonist, the remainder being suitable pharmaceutical carriers and/or diluents etc and optionally an opioid receptor agonist. Usually, a daily dose of the AT₁ receptor antagonist, candesartan, may be from about 1 to 40 mg per day, from about 4 to 20 mg or from 8 to 16 mg. The dosage of the AT₁ receptor antagonist can depend on a variety of factors, such as mode of administration, the species of the affected subject, age and/or individual condition. Normally, in the case of oral administration, an approximate daily dose of from about 4 mg to about 20 mg, for example in the case of candesartan of about 4 mg, 8 mg or 16 mg, is to be estimated for an adult patient of approximately 75 kg in weight.

In another embodiment, and dependent on the intended mode of administration, the opioid analgesic-containing compositions will generally contain about 0.1% to 90%, about 0.5% to 50%, or about 1% to about 25%, by weight of opioid analgesic, the remainder being suitable pharmaceutical carriers and/or diluents etc and optionally an AT₁ receptor antagonist. Usually, a daily oral dose of morphine in an opioid-naïve adult human may be from about 10 mg to 300 mg per day, from about 20 mg to 200 mg per day, or from about 30 mg to 180 mg per day. Generally, in the case of oral administration, an approximate daily dose of oxycodone in an opioid-naïve adult human may be from about 5 mg to about 200 mg, from about 10 mg to about 150 mg, or from about 20 mg to 100 mg per day, which is estimated for a patient of approximately 75 kg in weight.

Depending on the specific neuropathic condition being treated, the active compounds may be formulated and administered systemically, topically or locally. Techniques for formulation and administration may be found in “Remington's Pharmaceutical Sciences,” Mack Publishing Co., Easton, Pa., latest edition. Suitable routes may, for example, include oral, rectal, transmucosal, or intestinal administration; parenteral delivery, including intramuscular, subcutaneous, intramedullary injections, as well as intrathecal, direct intraventricular, intravenous, intraperitoneal, intranasal, or intraocular injections. For injection, the therapeutic agents of the invention may be formulated in aqueous solutions, suitably in physiologically compatible buffers such as Hanks' solution, Ringer's solution, or physiological saline buffer. For transmucosal administration, penetrants appropriate to the barrier to be permeated are used in the formulation. Such penetrants are generally known in the art.

Alternatively, the compositions of the invention can be formulated for local or topical administration. In this instance, the subject compositions may be formulated in any suitable manner, including, but not limited to, creams, gels, oils, ointments, solutions and suppositories. Such topical compositions may include a penetration enhancer such as benzalkonium chloride, digitonin, dihydrocytochalasin B, capric acid, increasing pH from 7.0 to 8.0. Penetration enhancers which are directed to enhancing penetration of the active compounds through the epidermis are preferred in this regard. Alternatively, the topical compositions may include liposomes in which the active compounds of the invention are encapsulated.

The compositions of this invention may be formulated for administration in the form of liquids, containing acceptable diluents (such as saline and sterile water), or may be in the form of lotions, creams or gels containing acceptable diluents or carriers to impart the desired texture, consistency, viscosity and appearance. Acceptable diluents and carriers are familiar to those skilled in the art and include, but are not restricted to, ethoxylated and nonethoxylated surfactants, fatty alcohols, fatty acids, hydrocarbon oils (such as palm oil, coconut oil, and mineral oil), cocoa butter waxes, silicon oils, pH balancers, cellulose derivatives, emulsifying agents such as non-ionic organic and inorganic bases, preserving agents, wax esters, steroid alcohols, triglyceride esters, phospholipids such as lecithin and cephalin, polyhydric alcohol esters, fatty alcohol esters, hydrophilic lanolin derivatives, and hydrophilic beeswax derivatives.

Alternatively, the active compounds of the present invention can be formulated readily using pharmaceutically acceptable carriers well known in the art into dosages suitable for oral administration, which is also preferred for the practice of the present invention. Such carriers enable the compounds of the invention to be formulated in dosage forms such as tablets, pills, capsules, liquids, gels, syrups, slurries, suspensions and the like, for oral ingestion by a patient to be treated. These carriers may be selected from sugars, starches, cellulose and its derivatives, malt, gelatine, talc, calcium sulphate, vegetable oils, synthetic oils, polyols, alginic acid, phosphate buffered solutions, emulsifiers, isotonic saline, and pyrogen-free water.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances that increase the viscosity of the suspension, such as sodium carboxymethyl cellulose, sorbitol, or dextran. Optionally, the suspension may also contain suitable stabilisers or agents that increase the solubility of the compounds to allow for the preparation of highly concentrated solutions.

Pharmaceutical preparations for oral use can be obtained by combining the active compounds with solid excipients, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as., for example, maize starch, wheat starch, rice starch, potato starch, gelatine, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Such compositions may be prepared by any of the methods of pharmacy but all methods include the step of bringing into association one or more therapeutic agents as described above with the carrier which constitutes one or more necessary ingredients. In general, the pharmaceutical compositions of the present invention may be manufactured in a manner that is itself known, eg. by means of conventional mixing, dissolving, granulating, dragee-making, levigating, emulsifying, encapsulating, entrapping or lyophilising processes.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterise different combinations of active compound doses.

Pharmaceuticals which can be used orally include push-fit capsules made of gelatine, as well as soft, sealed capsules made of gelatine and a plasticiser, such as glycerol or sorbitol. The push-fit capsules can contain the active ingredients in admixture with filler such as lactose, binders such as starches, and/or lubricants such as talc or magnesium stearate and, optionally, stabilisers. In soft capsules, the active compounds may be dissolved or suspended in suitable liquids, such as fatty oils, liquid paraffin, or liquid polyethylene glycols. In addition, stabilisers may be added.

Dosage forms of the active compounds of the invention may also include injecting or implanting controlled releasing devices designed specifically for this purpose or other forms of implants modified to act additionally in this fashion. Controlled release of an active compound of the invention may be achieved by coating the same, for example, with hydrophobic polymers including acrylic resins, waxes, higher aliphatic alcohols, polylactic and polyglycolic acids and certain cellulose derivatives such as hydroxypropylmethyl cellulose. In addition, controlled release may be achieved by using other polymer matrices, liposomes and/or microspheres.

The active compounds of the invention may be administered over a period of hours, days, weeks, or months, depending on several factors, including the severity of the neuropathic condition being treated, whether a recurrence of the condition is considered likely, etc. The administration may be constant, e.g., constant infusion over a period of hours, days, weeks, months, etc. Alternatively, the administration may be intermittent, e.g., active compounds may be administered once a day over a period of days, once an hour over a period of hours, or any other such schedule as deemed suitable.

The compositions of the present invention may also be administered to the respiratory tract as a nasal or pulmonary inhalation aerosol or solution for a nebuliser, or as a microfine powder for insufflation, alone or in combination with an inert carrier such as lactose, or with other pharmaceutically acceptable excipients. In such a case, the particles of the formulation may advantageously have diameters of less than 50 micrometers, suitably less than 10 micrometers.

In order that the invention may be readily understood and put into practical effect, particular preferred embodiments will now be described by way of the following non-limiting examples.

### EXAMPLES

### Example 1

Induction of STZ-Diabetes

Adult male Dark Agouti (DA) rats were obtained from the Central Animal Breeding House, The University of Queensland (Brisbane, Australia). The DA rat was utilised because in contrast to other rodent strains (for example, the Sprague-Dawley rat), it is genetically deficient in functional CYP2D1, thus conferring a negligible capacity to O-demethylate oxycodone to the potent μ-opioid agonist, oxymorphone (Cleary et al., 1994, J Pharmacol Exp Ther, 271: 1528-1534). This compares favourably with the low extent of CYP2D6-mediated O-demethylation of oxycodone to oxymorphone in humans (Al-Dabbagh et al., 1981, J Pharm Pharnacol, 33: 161-164; Zysset et al., 1988, Biochem Pharmacol, 37: 3155-3160) whereby <5% of each dose of oxycodone is O-demethylated to oxymorphone, resulting in extremely low circulating plasma concentrations of oxymorphone (Poyhia Ret al., 1991, Br J Clin Pharnacol, 32: 516-518; Poyhia et al., 1992, Br J Clin Pharnacol, 33: 617-621). Consequently, the DA rat is a closer animal model of the human for evaluating the antinociceptive/analgesic effects of oxycodone.

Rats were housed in solid floored cages with a layer of absorbent animal bedding which was changed on alternate days. The rats were kept in a room with a 12 h/12 h light/dark cycle at an ambient temperature of 21 (±2)° C. Standard rat chow and water were available ad libitum. Ethical approval for experiments described herein was obtained from the Animal Experimentation Ethics Committee of the University of Queensland.

Rats (220±20 g) were anaesthetised with a mixture of diazepam (3 mg/kg), ketamine (45 mg/kg) and xylazine (4 mg/kg) given by the intraperitoneal route to facilitate insertion of a polypropylene cannula (3 cm) into the jugular vein. Streptozotocin (STZ) (85 mg/kg freshly dissolved in 0.3 mL 20 mM sodium citrate buffer, pH 4.5) was then injected, the jugular vein cannula was removed and the vein tied off. Following closure of the surgical wound, antibiotic prophylaxis treatment was initiated in the form of topical antibiotic powder (neomycin sulfate, sulfacetamide sodium, nitrofurazone, phenylmercuric nitrate) over the sutured surgical incision and subcutaneously administered benzylpenicillin (60 mg). Ten days post-STZ administration, rats that drank in excess of 100 mL/day of water were classified as diabetic. This was confirmed by subsequent quantification of blood glucose concentrations—electrochemical detection using a MediSense 2 device (Waltham, Mass., USA). For the purposes of this study, hyperglycaemia was defined as blood glucose concentrations>15 mM, consistent with other studies in the literature (Courteix et al., 1994, Pain, 57: 153-160; Zurek et al., 2001 Pain 90: 57-63).

STZ-diabetic rats were allocated to one of three experimental groups: high-dose oral candesartan (2.0 mg/kg/day), low-dose oral candesartan (0.5 mg/kg/day) and STZ-diabetic controls.

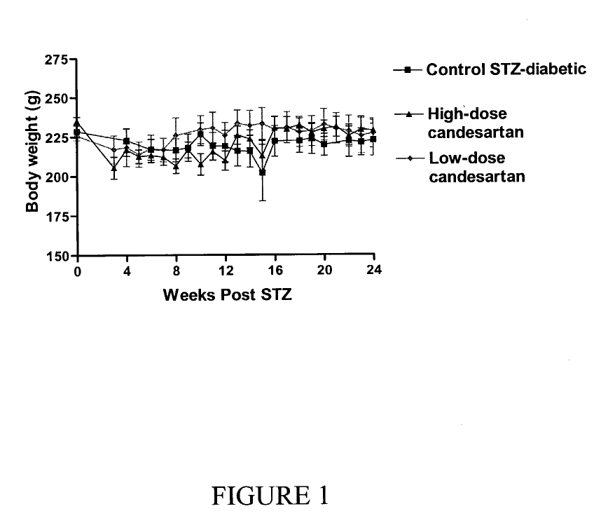

Of the 57 rats that received an intravenous (i.v.) bolus dose of STZ, 46 were successfully rendered diabetic, 6 failed to develop diabetes and the remaining 5 died within two wks of STZ administration. All STZ-diabetic rats exhibited hyperphagia (abnormally increased food intake) and polydipsia (abnormally increased thirst). Induction of STZ-diabetes produced an initial ≈5-10% reduction in (mean±SEM) body weight from 233 (±1.99) g pre-STZ administration to 218 (±3.28) g at 4 wks post-STZ, followed by a gradual return to the pre-study weight over the ensuing 6-mth study period (FIG. 1). Blood glucose concentrations in diabetic rats were significantly increased relative to the respective concentrations in non-diabetic rats with blood glucose concentrations exceeding 20 mM by 3 wks-post-STZ administration (Table 1). None of these above parameters were significantly altered by chronic once-daily oral administration of candesartan at either of the doses investigated.

### Example 2

Effect of Prophylactic Candesartan on the Development of Tactile Allodynia in STZ-Diabetic Rats

Candesartan Prevention Protocol

Candesartan treatment was initiated after STZ administration (day 0) but prior to the onset of diabetes. Specifically, candesartan-treated rats received this drug via once-daily oral gavage in one of two dosages: viz a high-dose (2 mg/kg/day) or a low dose (0.5 mg/kg/day). The 2 mg/kg/day dose was chosen on the basis that it is an anti-hypertensive dose both in rats with perfused Ang II-induced hypertension and also in the spontaneously hypertensive rat (SHR) (Nishikawa et al., 1994, Blood Press, Suppl 5: 7-14). The low-dose (0.5 mg/kg/mg) was included to investigate whether a sub-therapeutic dose (in terms of anti-hypertensive activity) of candesartan was efficacious for the treatment of PDN. Once initiated, oral once-daily candesartan was administered every day for 24 wks.

Three control experimental groups were also studied, including age-matched STZ-diabetic rats that did not receive candesartan (control STZ-diabetic rats). A second group of STZ-diabetic rats received vehicle (DMSO:water 10:90) by oral gavage (protocol-control rats). In the third group, weight-matched, naive, non-diabetic control rats (non-diabetic candesartan control rats) were also treated with candesartan for one wk prior to opioid antinociceptive testing to determine any intrinsic effects of candesartan on baseline pain scores and on opioid-mediated antinociception. Weight-matched (as opposed to age-matched) naïve controls were employed on the basis that weight-matched controls more closely reflect the pharmacokinetics of diabetic animals due to similar proportions of subcutaneous body fat. In comparison, age-matched controls would be expected to differ significantly in terms of their volume of distribution of opioid drugs, due to their much higher proportion of body fat. Moreover, as the absolute body weight of age-matched control non-diabetic rats is almost twice that of age-matched control diabetic rats, it is more relevant to use weight-matched non-diabetic rats for studies involving opioid dosing on a mg/kg basis.

Von Frey Assessment of Tactile Allodynia

Tactile allodynia, the defining symptom of PDN, manifests as a hypersensitive response to non-noxious stimuli such as touch or light pressure, and was quantified using Von Frey filaments (VFFs). By contrast, many previous studies have quantified NCV as an index of PDN in both humans and experimental animals (Maxfield et al., 1993, supra; Cameron et al., 1994, 37: 1209-1215; Malik et al., 1998, supra; Cameron and Cotter, 1999, Diabetes Res Clin Pract, 45: 137-146; van Dam et al., 1999, Eur J Pharmacol, 376: 217-222; Zochodne & Nguyen, 1999, J Neurol Sci, 166: 40-46). However, indirect methods have a questionable correlation with symptomatic severity (Malik et al., 1998, supra; Malik et al., 2001, supra). Therefore direct quantification of symptom severity using VFF assessment was used to obtain clinically relevant end points.

For each antinociceptive testing session, rats were placed in wire mesh metabolic cages (20 cm×20 cm×20 cm) and allowed to acclimatise to the test environment for approximately 10-15 min prior to the commencement of experiments. Calibrated VFFs, delivering a force in the range 2-20 g, were then applied to the plantar surface of the hind-paw to determine the paw withdrawal thresholds defined as the minimum force necessary to elicit a brisk foot withdrawal reflex. Commencing with the VFF delivering the lowest mechanical force, the filament was applied to the plantar surface of the footpad until buckling of the filament was observed. Absence of a response after approximately 3 s prompted application of the next VFF of increasing force to the footpad. Rats exhibiting no response after application of the VFF that delivered the 20 g force, were arbitrarily assigned a paw withdrawal threshold of 20 g.

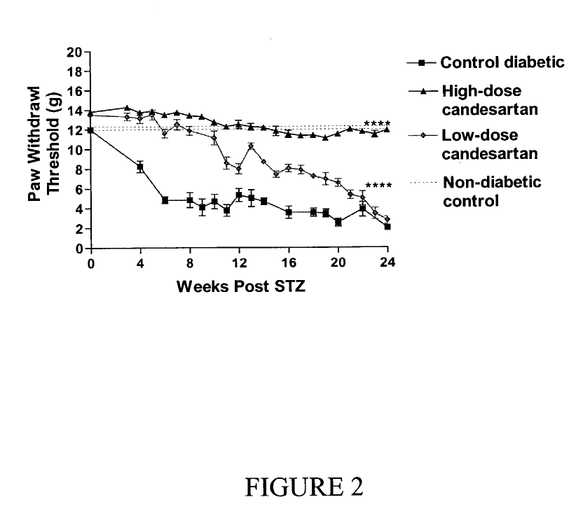

The onset and progression of the development of tactile allodynia in all STZ-diabetic rats were quantified by once weekly VFF paw withdrawal testing. Specifically for each rat, three separate assessments of paw withdrawal thresholds were undertaken, each approximately 5 min apart. The paw withdrawal thresholds assessed using VFFs for each of the experimental groups of diabetic rats are shown in FIG. 2.

Control STZ-Diabetic Rats

There was a marked temporal decrease in Von Frey paw withdrawal thresholds in control STZ-diabetic rats, such that the mean (±SEM) paw withdrawal threshold decreased from 12.14 (±0.15) g pre-STZ administration to 8.25 (±0.59) g at 4 wks post-STZ and 4.83 (±0.33) g at 6 wks post-STZ. Thereafter, mean (±SEM) paw withdrawal thresholds remained relatively stable until approximately 16 wks before gradually decreasing to a mean (±SEM) value of 2.40 (±0.40) g at 24 wks post-STZ. These findings show that the development of tactile allodynia (the defining symptom of PDN) was maintained throughout the 6-mth post-STZ study period.

Oral Candesartan Administration

Once-daily oral administration of candesartan attenuated the development of tactile allodynia in a dose-dependent manner. Most notably, chronic administration of oral candesartan at an anti-hypertensive dose (2.0 mg/kg/day) completely attenuated the development of tactile allodynia as assessed by Von Frey filaments (p<0.0001), such that the mean (±SEM) paw withdrawal threshold at 24 wks post-STZ administration was not significantly different (p>0.05) from that seen in non-diabetic control rodents. Although STZ-diabetic rats that received chronic once-daily oral administration of a sub-anti-hypertensive dose of candesartan (0.5 mg/kg/day) had similar paw withdrawal thresholds to rats that received the higher dose of candesartan for the first 10 wks post-STZ, this effect was not maintained. By 22 wks post-STZ, the paw withdrawal thresholds were not significantly different (p>0.05) from those observed in untreated STZ-diabetic control rats, indicating that low-dose candesartan only delayed but did not prevent the development of tactile allodynia.

### Example 3

Opioid-Mediated Antinociception

The antinociceptive potencies of oxycodone and morphine for the relief of tactile allodynia were determined in all treatment groups. While full dose-response curves for each of subcutaneous (s.c.) morphine and oxycodone were determined in high-dose candesartan-treated STZ-diabetic rats and the candesartan-treated non-diabetic control rats, untreated STZ-diabetic rats and the low-dose candesartan-treated rats received single s.c. bolus doses (≈ED₅₀) each of oxycodone and morphine at 3, 9, 12 and 24 wks post-STZ administration. In all cases, opioids were administered by a single s.c. injection (100 μL) into the dorsal region at the base of the neck whilst under light CO₂/O₂ (50:50%) anaesthesia using a 250 μL Hamilton syringe. For the STZ-diabetic treatment groups, the antinociceptive effects of morphine and oxycodone were determined at 3, 9, 12 and 24 wks, and at 3, 9 and 24 wks post-STZ administration, respectively. By contrast, the opioid-naïve, weight-matched, non-diabetic candesartan control rats were given high-dose oral candesartan (2 mg/kg/day) for 7 days before opioid testing was initiated. Additionally, for all experimental groups, rats administered with either s.c. morphine or oxycodone were allowed a 3 day wash-out period prior to a crossover opioid antinociceptive testing session with the alternative opioid.

Immediately prior to administration of s.c. bolus doses of either opioid, baseline paw withdrawal thresholds were quantified using VFFs in an identical manner to the weekly baseline Von Frey monitoring described above. Following s.c. opioid administration, VFF assessments were performed at the following post-dosing times: 15, 30, 45, 60, 90, 120 and 180 min.

Materials

Oxycodone hydrochloride was a generous gift from Tasmanian Alkaloids Pty Ltd (Hobart, Australia). Morphine hydrochloride and diazepam (Valium®) was obtained from the Pharmacy Department, Royal Brisbane Hospital (Brisbane, Australia). Streptozotocin (STZ), dimethyl sulfoxide (DMSO), citric acid and trisodium citrate were purchased from Sigma Chemical Company (Sydney, Australia). Sodium benzylpenicillin (BenPen™), ketamine (Ketamav™) and xylazine (Xylazil™) were purchased from Abbott Australasia Pty Ltd (Sydney, Australia). Topical antibiotic powder was purchased from Apex Laboratories Pty Ltd (Somersby, Australia). Medical grade O₂ and CO₂ were purchased from BOC Gases Australia Ltd (Brisbane, Australia). Blood glucose sensor electrodes (MediSense®) were purchased from Abbott Laboratories (Bedford, United Kingdom). Morphine hydrochloride and oxycodone hydrochloride were dissolved in isotonic saline and stored at −4° C. until required. Similarly, candesartan cilexetil was prepared in a mixture of DMSO (10%) and deionised water (90%) and stored at −4° C. until required.

Data Analysis

The VFF scores for individual rats were converted to the Percentage of the Maximum Possible Antinociceptive Effect (% MPE), according the following formula (Brady & Holtzmann, 1982):

$$\begin{matrix}\% MPE = \frac{( Post Drug Threshold - Predrug Threshold )}{( Maximum threshold - Predrug Threshold )} \times \frac{100}{1} \\ where maximum VFF threshold = 20 g\end{matrix}$$

The area under the % MPE versus time curve from time=0-3h (% MPE AUC) was estimated using trapezoidal integration. The mean (±SEM) percentage maximum AUC (% Max AUC) was calculated according to the following formula:

$$\begin{matrix}\% Max AUC = \frac{\% MPE AUC}{Maximum \% MPE AUC} \times \frac{100}{1} \\ where maximum \% MPE AUC = 263 \% MPE \cdot h\end{matrix}$$

The % Max AUC for each of morphine or oxycodone was plotted versus the respective drug dose to produce individual opioid dose-response curves. ED₅₀ doses (mean±SEM) for each of morphine and oxycodone were estimated using non-linear regression of the % Max AUC versus log dose values, as implemented in Graphpad Prism™. ED₅₀ estimation was facilitated by inclusion of the theoretical maximum and minimum % Max AUC values. The Mann-Whitney test was used to compare %MPE AUC ED₅₀ values between treatment groups. The statistical significance criterion was p<0.05.

Control STZ-Diabetic Rats

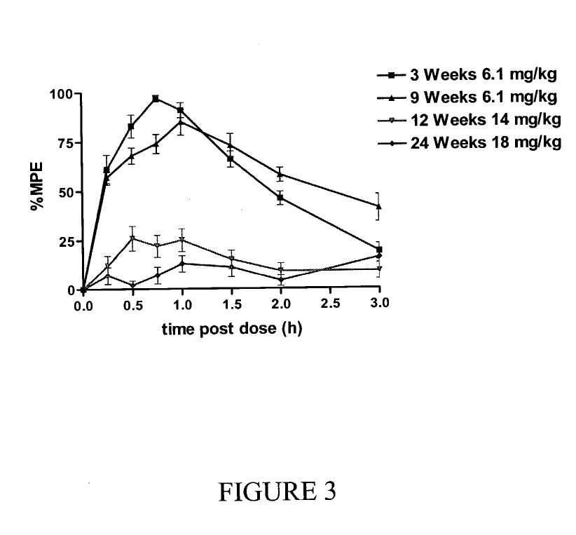

Morphine (FIG. 3 and Table 2)

The extent and duration of the antinociceptive response (%MPE AUC) evoked by bolus doses (ED₅₀) of s.c. morphine in control STZ-diabetic rats did not differ significantly (p>0.05) between 3 and 9 wks post-STZ administration (Table 2). A small alteration of the timing of the peak antinociceptive effect was evident (FIG. 3), shifting from approximately 45 min at 3 wks post-STZ to 60 min at 9 wks post-STZ (p<0.01).

Consistent with recent findings by the inventors (Smith et al., 2001, Proceedings of the Australasian Society of Clinical and Experimental Pharmacologists and Toxicologists, 9: 38), that the antinociceptive efficacy of s.c. morphine in control STZ-diabetic rats was completely abolished by 12 wks post-STZ administration, there was also a marked decrease in the antinociceptive response evoked by morphine in the control STZ-diabetic rats used in the present studies.

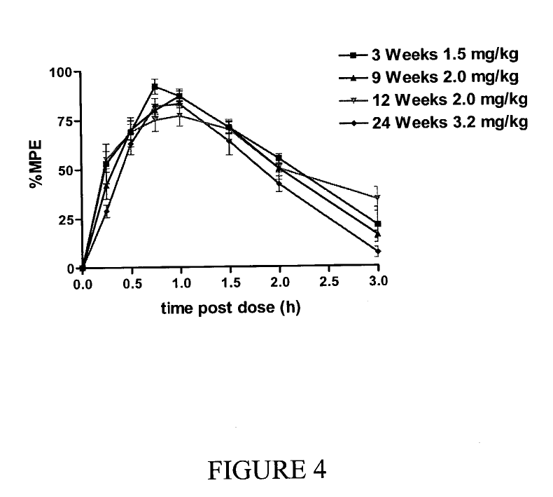

Oxycodone (FIG. 4 and Table 3)

By contrast with morphine, a recent study has shown that the full antinociceptive efficacy of bolus doses of s.c. oxycodone is maintained in STZ-diabetic rats throughout the 24 wk study period, albeit with a 4-fold decrease in potency relative to non-diabetic control rats (Smith et al., 2001, supra). Data herein show that there was an approximately 3-fold decrease in potency at 24 wks post-STZ administration relative to protocol control STZ-diabetic rats. Additionally, the mean (±SEM) time to achieve peak antinociception did not change significantly (p>0.05) over the same study period.

Candesartan (2.0 mg/kg/day) Treated STZ-Diabetic Rats

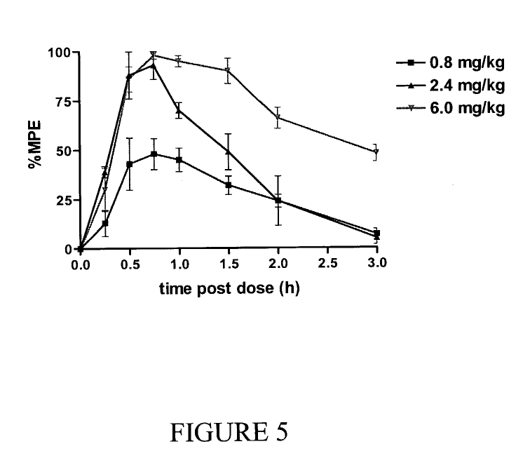

Morphine (FIGS. 5-7 and Table 4)

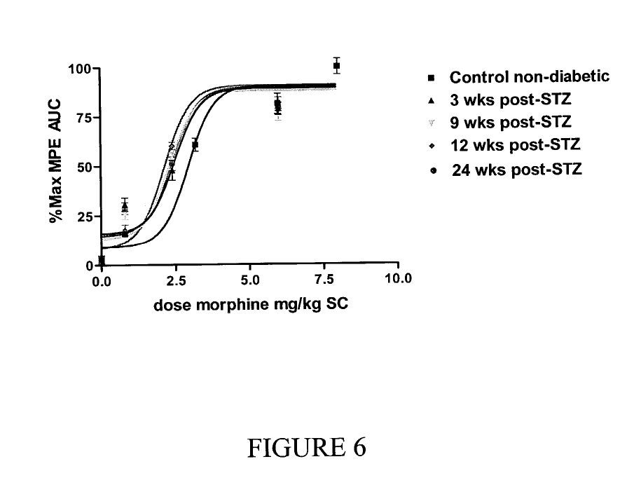

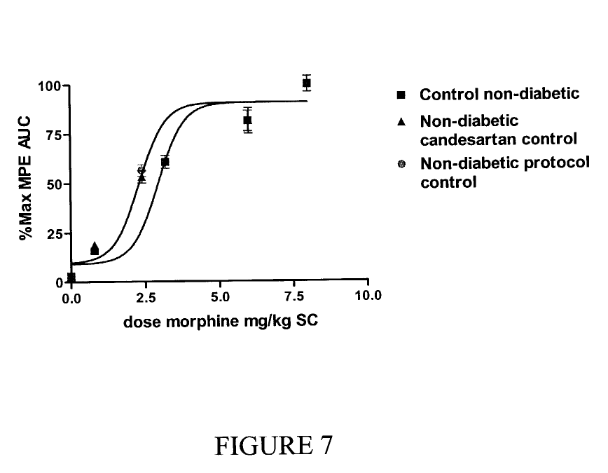

Remarkably, chronic once-daily oral administration of an anti-hypertensive dose of candesartan (2.0 mg/kg/day) to STZ-diabetic rats preserved the antinociceptive potency of morphine for the full 24 wk duration of the study, such that the ED₅₀ values at 24 wks post-STZ rats (ED₅₀=2.4 mg/kg) was not significantly different (p>0.05) from that in non-diabetic protocol-control rats (Table 4). Examination of the s.c. morphine dose-response curves in high-dose oral candesartan-treated STZ-diabetic rats determined at 3, 9, 12 and 24 wks post-STZ (FIG. 6), revealed that once-daily oral administration of candesartan at an anti-hypertensive dose (2 mg/kg/day) completely prevented the temporal loss of morphine potency and efficacy throughout the 24 wk post-STZ study period relative to non-diabetic control rats. This finding contrasts with the distinct temporal loss of morphine potency and efficacy in untreated STZ-diabetic control rats. Additionally, this preserving effect of high-dose oral candesartan occurred independent of any direct alterations upon morphine pharmacology as the morphine dose-response curve in control non-diabetic rats that received chronic once-daily high-dose oral candesartan treatment (2.0 mg/kg/day) was not significantly different (p>0.05) from that for non-diabetic control rats that did not receive oral candesartan treatment (Table 5 and FIG. 7).

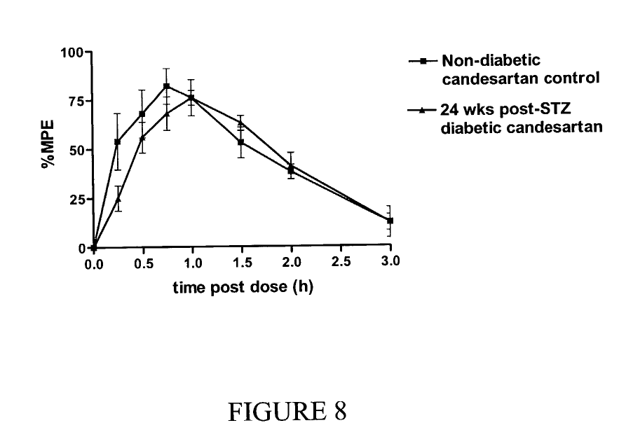

The mean (±SEM) time to reach peak levels of antinociception following bolus doses of s.c. morphine however, increased significantly from 45 min at 3 wks post-STZ to 60 min beyond 12 wks post-STZ administration (p<0.05) in high-dose oral candesartan-treated STZ-diabetic rats. Comparison of the mean (±SEM) degree of antinociception (%MPE) versus time curves between non-diabetic and 24 wk post-STZ diabetic rats (FIG. 8) that both received anti-hypertensive doses of candesartan (2.0 mg/kg/day), indicates that this increase in the time to reach peak morphine antinociception occurred independent of candesartan treatment and is attributable largely to the diabetic state.

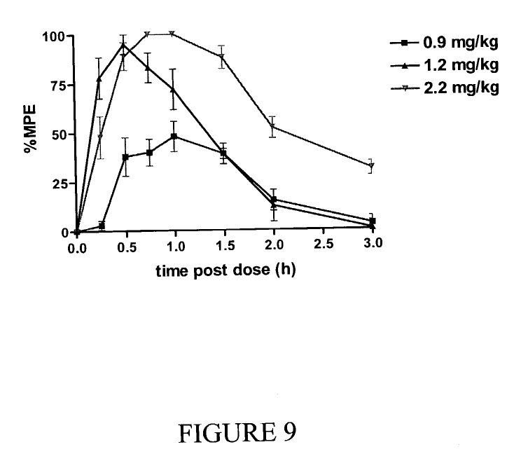

Oxycodone (FIGS. 9-11 and Tables 6-7)

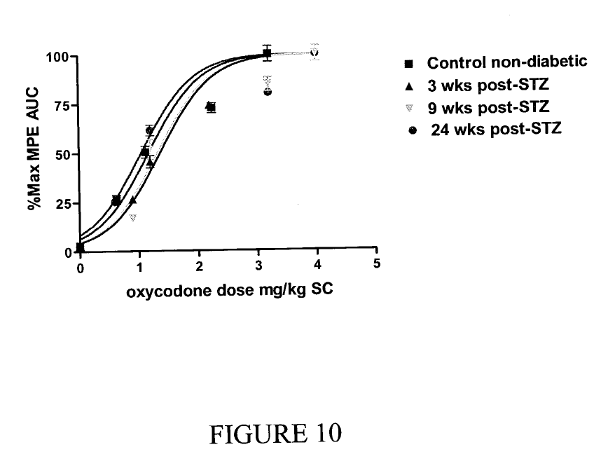

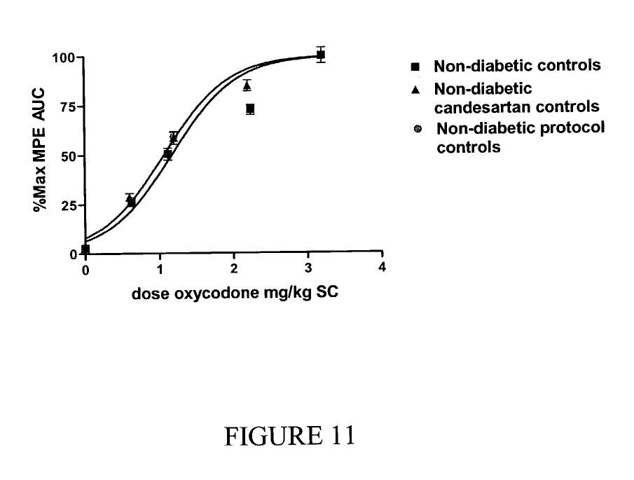

The potency of oxycodone in STZ-diabetic rats was similarly preserved by once-daily oral administration of an anti-hypertensive dose of candesartan (2.0 mg/kg/day) (Table 6) with no significant alterations (p>0.05) in the timing for peak antinociceptive effect during the 24 wk experimental period. Inspection of the dose-response curves for s.c. oxycodone in these high-dose oral candesartan-treated STZ-diabetic rats determined at 3, 9 and 24 wks post-STZ (FIG. 9), again revealed that once-daily oral administration of candesartan at an anti-hypertensive dose (2 mg/kg/day) completely prevented the temporal loss of oxycodone potency and efficacy throughout the 24 wk post-STZ study period relative to non-diabetic control rats (FIG. 10). As in the case for morphine, the protective effect of high-dose candesartan on oxycodone potency in STZ-diabetic rats occured independent of direct alterations by oral candesartan upon s.c. oxycodone pharmacology as the dose-response curve for s.c. oxycodone in high-dose oral candesartan-treated (2.0 mg/kg/day) non-diabetic control rats was not significantly different (p>0.05) relative to that for non-diabetic control rats not receiving candesartan treatment (Table 7 and FIG. 11). Taken together, these data show that oral administration of high-dose candesartan in STZ-diabetic rats prevents the 3-fold decrease in the antinociceptive potency of oxycodone previously observed in control STZ-diabetic rats across the 24 wk post-STZ study period.

Candesartan (0.5 mg/kg/day) Treated STZ-Diabetic Rats

Morphine (Table 8)

Chronic once-daily oral administration of a sub-anti-hypertensive dose of candesartan (0.5 mg/kg/day) to STZ-diabetic rats attenuated the loss of morphine potency relative to the complete abolition of morphine's antinociceptive efficacy observed in control untreated STZ-diabetic rats. Although low-dose candesartan maintained morphine's full antinociceptive efficacy throughout the 24 wk post-STZ study period, a distinct and statistically significant (p<0.01) loss of antinociceptive potency was apparent at 12 wks post-STZ and beyond, as illustrated by the significant decrease in the dose-normalised %MPE AUC values (Table 6).

Oxycodone (Table 9)

The antinociceptive efficacy of oxycodone in STZ-diabetic rats that received once-daily oral administration of a sub-anti-hypertensive dose of candesartan (0.5 mg/kg/day) was maintained throughout the duration of the study with no significant temporal shift in the mean (±SEM) time to reach peak levels of antinociception. Inspection of the dose-normalised %MPE AUC values however, revealed a decline in the potency of oxycodone over the 24 wk study period, such that by 24 wks post-STZ administration there was an approximate 1.5-fold decrease in the dose-normalised %MPE AUC values for oxycodone in STZ-diabetic rats that received low-dose candesartan when compared to protocol-control non-diabetic rats.

### Example 4

Reversal Protocol: Pilot Study

Chronic High-Dose Candesartan Treatment: Cessation and Re-initiation

Cessation of candesartan treatment in six 24 wks post-STZ rats that previously received chronic once-daily high-dose cadesartan treatment (2.0 mg/kg/day) resulted in an apparent general decline in the physical appearance and health of the STZ-diabetic rats. One rat died 5 wks after the cessation of candesartan therapy. Re-initiation of once-daily oral high-dose candesartan therapy in the remaining five rats after a 6-wk interval reversed these behavioural changes.

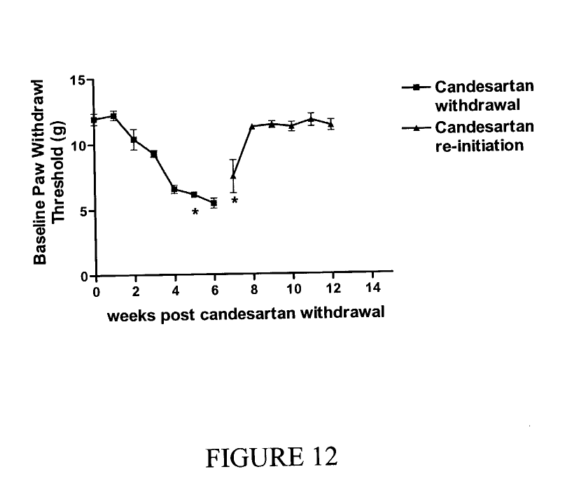

Effect on Von Frey Baseline Paw Withdrawal Thresholds (FIG. 12)

Cessation of chronic high-dose oral candesartan treatment resulted in a decrease in the mean (±SEM) paw withdrawal threshold from 11.9 (±0.2) g prior to cessation of candesartan therapy at 24 wks post-STZ, to 6.0 (±0.3) g after six wks. Re-initiation of once-daily high-dose oral candesartan (2.0 mg/kg/day) administration restored the paw withdrawal thresholds within two wks to levels (11.3±0.1 g) not significantly different (p>0.05) from values observed in the same rats immediately prior to candesartan cessation (11.9±0.2 g) and not significantly different from paw withdrawal thresholds found in non-diabetic control rats (12.1±0.2 g).

By contrast, chronic once-daily oral administration of vehicle (10% DMSO in water) in STZ-diabetic rats beyond 12 wks post-STZ for 2 wks did not significantly restore paw withdrawal thresholds.

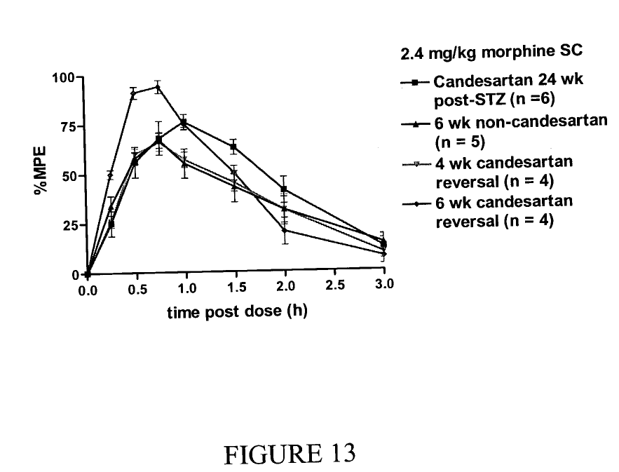

Effect on Morphine Antinociception (FIG. 13 and Table 10)

Although cessation of once-daily high-dose oral candesartan treatment (2.0 mg/kg/day) appeared to result in a temporal loss of morphine potency, such that there was a trend for the mean (±SEM) area under the %MPE versus time curve evoked by s.c. morphine (2.4 mg/kg) to decrease from 135±9.8%MPE.h in 24 wks post-STZ rats administered high-dose candesartan to 112±14.2%MPE.h at 6 wks after candesartan cessation, this apparent decrease did not reach statistical significance. Importantly, re-initiation of once-daily oral candesartan (2.0 mg/kg/day) completely reversed this trend such that the mean (±SEM) area under the %MPE versus time curve for morphine after 6 wks of treatment (%MPE AUC=130±7.5%MPE AUC.h) was very similar to that observed prior to cessation of high-dose candesartan.

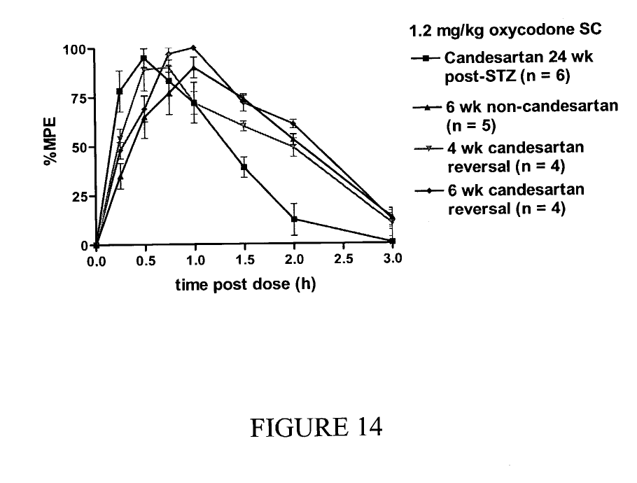

Effect on Oxycodone Antinociception (FIG. 14 and Table 11)

Cessation of once-daily oral administration of high-dose candesartan (2.0 mg/kg/day) resulted in a small but insignificant decrease in the potency of s.c. oxycodone, such that the mean area under the %MPE versus time curve following administration of s.c. oxycodone decreased from 162±7.1%MPE.h in 24 wks post-STZ diabetic rats receiving high-dose oral candesartan to 139±11.5%MPE.h at 6 wks after cessation of candesartan treatment in the same rats. This decrease however, was completely reversed 6 wks after re-initiation of chronic high-dose oral candesartan (2.0 mg/kg/day) (%MPE AUC=179±5.4%MPE.h).

### Example 5

Reversal Protocol: Pilot Study

Induction of STZ-Diabetes

Adult male Dark Agouti (DA) rats were obtained from the Central Animal Breeding House, The University of Queensland (Brisbane, Australia). Rats were housed in solid floored cages with a layer of absorbent animal bedding which was changed on alternate days. The rats were kept in a room with a 12 h/12 h light/dark cycle at an ambient temperature of 21 (±2)° C. Standard rat chow and water were available ad libitum. Ethical approval for experiments described herein was obtained from the Animal Experimentation Ethics Committee of the University of Queensland.

The DA rats (220±20 g) were anaesthetised with a mixture of diazepam (3 mg/kg), ketamine (45 mg/kg) and xylazine (4 mg/kg) given by the intraperitoneal route to facilitate insertion of a polypropylene cannula (≈3 cm) into the jugular vein. Streptozotocin (STZ) (85 mg/kg freshly dissolved in 0.3 mL 20 mM sodium citrate buffer, pH 4.5) was then injected, the jugular vein cannula was removed and the vein tied off. Following closure of the surgical wound, antibiotic prophylaxis treatment was initiated in the form of topical antibiotic powder (neomycin sulfate, sulfacetamide sodium, nitrofurazone, phenylmercuric nitrate) over the sutured surgical incision and subcutaneously administered benzylpenicillin (60 mg). Ten days post-STZ administration, rats that drank in excess of 100 mL/day of water were classified as diabetic. This was confirmed by subsequent quantification of blood glucose concentrations—electrochemical detection using a MediSense 2 device (Waltham, Mass., USA). For the purposes of this study, hyperglycaemia was defined as blood glucose concentrations >15 mM, consistent with other studies in the literature (Courteix et al., 1994, Pain, 57: 153-160; Zurek et al., 2001 Pain 90: 57-63).

Von Frey Assessment of Tactile Allodynia

Tactile allodynia, the defining symptom of PDN, manifests as a hypersensitive response to non-noxious stimuli such as touch or light pressure, and was quantified using Von Frey filaments (VFFs). By contrast, many previous studies have quantified NCV as an index of PDN in both humans and experimental animals (Maxfield et al., 1993, supra; Cameron et al., 1994, 37: 1209-1215; Malik et al., 1998, supra; Cameron and Cotter, 1999, Diabetes Res Clin Pract, 45: 137-146; van Dam et al., 1999, Eur J Pharmacol, 376: 217-222; Zochodne & Nguyen, 1999, J Neurol Sci, 166: 40-46). However, indirect methods have a questionable correlation with symptomatic severity (Malik et al., 1998, supra; Malik et al., 2001, supra). Direct quantification of symptom severity using VFF assessment, has the potential to yield more clinically relevant end points.

For each antinociceptive testing session, rats were placed in wire mesh metabolic cages (20 cm×20 cm×20 cm) and allowed to acclimatise to the test environment for approximately 10-15 min prior to the commencement of experiments. Calibrated VFFs, delivering a force in the range 2-20 g, were then applied to the plantar surface of the hind-paw to determine the paw withdrawal thresholds defined as the minimum force necessary to elicit a brisk foot withdrawal reflex. Commencing with the VFF delivering the lowest mechanical force, the filament was applied to the plantar surface of the footpad until buckling of the filament was observed. Absence of a response after approximately 3 s prompted application of the next VFF of increasing force to the footpad. Rats exhibiting no response after application of the VFF that delivered the 20 g force, were arbitrarily assigned a paw withdrawal threshold of 20 g.

The onset and progression of the development of tactile allodynia in all STZ-diabetic rats were quantified by periodic VFF paw withdrawal testing. Specifically for each rat, three separate assessments of paw withdrawal thresholds were undertaken, each approximately 5 min apart.

Once-Daily AT1 Antagonist Treatment: Reversal Protocol

STZ-diabetic rats (n=18) were allocated to one of three experimental groups. Groups one and two received once-daily oral administration of antihypertensive doses of one of the AT1-antagonists, viz candesartan (2.0 mg/kg/day)or losartan (20 mg/kg/day), commencing at 12 weeks post-STZ administration (day 0). The third group (control STZ-diabetic rats) received no treatment.

Treatment with an AT1 Antagonist: Reversal Protocol

Treatment of STZ-diabetic rats with either once-daily oral candesartan (2 mg/kg/day) or once-daily oral losartan (20 mg/kg/day) was initiated 12 weeks after STZ-administration (day 0), i.e. the candesartan or losartan treatments were not initiated until the defining symptom of PDN (tactile allodynia) had been fully developed for more than 8 weeks. Specifically, at 12 weeks post-STZ administration, the mean (±SEM) baseline paw withdrawal threshold prior to initiation of candesartan treatment was 2.9 (±0.3) g whereas the mean (±SEM) baseline paw withdrawal threshold prior to administration of STZ was 12.1 (±0.2) g.

Opioid-Mediated Antinociception

The antinociceptive potency of a single bolus dose of s.c. morphine (6.1 mg/kg) for the relief of tactile allodynia was determined. Morphine was administered by a single s.c. injection (100 μL) into the dorsal region at the base of the neck whilst under light CO₂/O₂ (50:50%) anaesthesia using a 250 μL Hamilton syringe. Immediately prior to administration of s.c. bolus doses of morphine, baseline paw withdrawal thresholds were quantified using VFFs in an identical manner to the baseline Von Frey monitoring described above. Following s.c. opioid administration, VFF assessments were performed at the following post-dosing times: 15, 30, 45, 60, 90, 120 and 180 min.

Materials

Morphine hydrochloride and diazepam (Valium®) was obtained from the Pharmacy Department, Royal Brisbane Hospital (Brisbane, Australia). Streptozotocin (STZ), dimethyl sulfoxide (DMSO), citric acid and trisodium citrate were purchased from Sigma Chemical Company (Sydney, Australia). Sodium benzylpenicillin (BenPen™), ketamine (Ketamav™) and xylazine (Xylazil™) were purchased from Abbott Australasia Pty Ltd (Sydney, Australia). Topical antibiotic powder was purchased from Apex Laboratories Pty Ltd (Somersby, Australia). Medical grade O₂ and CO₂ were purchased from BOC Gases Australia Ltd (Brisbane, Australia). Blood glucose sensor electrodes (MediSense®) were purchased from Abbott Laboratories (Bedford, United Kingdom). Morphine hydrochloride was dissolved in isotonic saline and stored at −4° C. until required. Similarly, candesartan cilexetil was prepared in a mixture of DMSO (10%) and deionised water (90%) and stored at −4° C. until required. Losartan potassium was extracted from Cozaar™ tablets and then dissolved in deionised water just prior to administration.

Data Analysis

The VFF scores for individual rats were converted to the Percentage of the Maximum Possible Antinociceptive Effect (% MPE), according the following formula (Brady & Holtzmann, 1982):

$$\begin{matrix}\% MPE = \frac{( Post Drug Threshold - Predrug Threshold )}{( Maximum threshold - Predrug Threshold )} \times \frac{100}{1} \\ where maximum VFF threshold = 20 g\end{matrix}$$

The area under the % MPE versus time curve from time=0-3h (% MPE AUC) was estimated using trapezoidal integration. The mean (±SEM) percentage maximum AUC (% Max AUC) was calculated according to the following formula:

$$\begin{matrix}\% Max AUC = \frac{\% MPE AUC}{Maximum \% MPE AUC} \times \frac{100}{1} \\ where maximum \% MPE AUC = 263 \% MPE \cdot h\end{matrix}$$

The % Max AUC (i.e. %Max response) for morphine was plotted versus the number of weeks of STZ-diabetes to produce a response versus time curve. The Mann-Whitney test was used to compare %MAX AUC values between treatment groups. The statistical significance criterion was p<0.05.

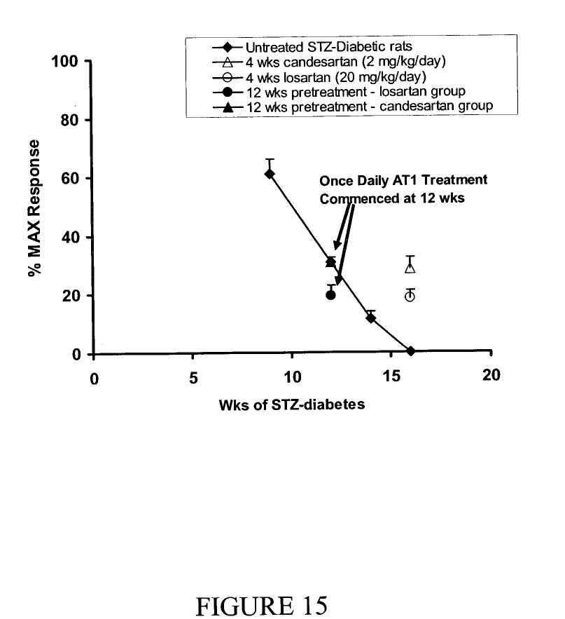

Morphine (FIG. 15)

For control STZ-diabetic rats that received no pharmacological interventions, the antinociceptive potency of bolus s.c. doses of morphine (6.1 mg/kg) decreased in a temporal manner such that antinociceptive efficacy was abolished by 16 wks post-STZ administration. By contrast, 4 wks of either once-daily candesartan (2 mg/kg/day) or losartan (20 mg/kg/day) given by oral gavage, commencing at 12 wks post-STZ administration, preserved morphine's antinociceptive effects. Specifically at 16-wks post-STZ administration in rats that had received 4 wks of once daily oral candesartan (2 mg/kg/day) treatment, the antinociceptive potency of single bolus doses of s.c. morphine (6.1 mg/kg) did not differ significantly (p>0.05) from that determined in the same STZ-diabetic rats at 12 wks post-STZ, prior to initiation of candesartan treatment. Similarly, for rats that received treatment with once-daily oral losartan (20 mg/kg/day), the antinociceptive potency of single bolus doses of s.c. morphine (6.1 mg/kg), did not differ significantly from that determined in the same STZ-diabetic rats prior to initiation of losartan treatment at 12 wks post-STZ.

These data show that 4 wks of either once-daily candesartan or losartan administration by oral gavage, commencing at 12 wks post-STZ administration, preserved morphine's antinociceptive effects.

The disclosure of every patent, patent application, and publication cited herein is hereby incorporated herein by reference in its entirety.

The citation of any reference herein should not be construed as an admission that such reference is available as “Prior Art” to the instant application

Throughout the specification the aim has been to describe the preferred embodiments of the invention without limiting the invention to any one embodiment or specific collection of features. Those of skill in the art will therefore appreciate that, in light of the instant disclosure, various modifications and changes can be made in the particular embodiments exemplified without departing from the scope of the present invention. All such modifications and changes are intended to be included within the scope of the appended claims.

Tables

TABLE 1
Mean (±SEM) blood glucose concentrations in non-diabetic and STZ-diabetic rats.

| | | | Mean (±SEM) Blood |
|---|---|---|---|
| | Treatment | | Glucose Concentration |
| EXPERIMENTAL GROUP | Description | n | (mM) |
| Non-diabetic control rats | No treatment | 6 | 5.9 (±0.3) |
| Non-diabetic protocol control rats | 2 wks treatment | 6 | 7.7 (±0.5) |
| Non-diabetic high-dose oral candesartan | 2 wks treatment | 6 | 6.2 (±0.3) |
| (2.0 mg/kg/day) control rats | | | |
| Control STZ-diabetic rats | 12 wks post-STZ | 6 | 21.0 (±1.0.) |
| | 24 wks post-STZ | 6 | 23.7 (±0.6) |
| High-dose oral candesartan | 3 wks post-STZ | 6 | 20.7 (±0.9) |
| (2.03 mg/kg/day) STZ-diabetic rats | 9 wks post-STZ | 4 | 24.8 (±1.1) |
| | 12 wks post-STZ | 6 | 21.4 (±1.1) |
| | 24 wks post-STZ | 12 | 20.94 (±0.6) |
| Low-dose oral candesartan | 9 wks post-STZ | 6 | 22.3 (±1.1) |
| (0.5 mg/kg/day) STZ-diabetic rats | 12 wks post-STZ | 6 | 21.9 (±0.7) |
| | 24 wks post-STZ | 6 | 22.3 (±1.2) |

TABLE 2
Temporal change in the mean (±SEM) area under the decree of
antinociception (expressed as the % maximum possible effect, % MPE)
versus time curve (% MPE AUC values) following s.c.
administration of bolus doses of morphine in control STZ-diabetic rats.

| | | | Mean | Mean (±SEM) | |
|---|---|---|---|---|---|
| | | | (±SEM) | dose normalised | |
| Wks | | Morphine | % MPE | % MPE AUC | |
| Post- | | Dose | AUC | (% MPE | |
| STZ | n | (mg/kg) | (% MPE.h) | AUC.h.kg.mg⁻¹) | Significance |
| Protocol | 6 | 2.4 | 149 (±18.8) | 62.0 (±3.2) | |
| Control | | | | | |
| 3 | 6 | 6.1 | 171 (±5.9) | 28.0 (±1.0) | p < 0.01 |
| 9 | 6 | 6.1 | 182 (±9.1) | 29.8 (±1.5) | p < 0.01 |
| 12 | 6 | 14 | 34 (±11.4) | 2.5 (±0.8) | p < 0.01 |
| 24 | 5 | 18 | 26 (±4.5) | 1.4 (±0.2) | p < 0.01 |

At 3 and 9 wks post-STZ, diabetic rats received the ED₅₀ morphine dose (6.1 mg/kg) previously determined by Smith et al. (2001, Proceedings of the Australasian Society of Clinical and Experimental Phannacologists and Toxicologists, 9: 38). The morphine dose was increased to 14 mg/kg and 18 mg/kg at 12 and 24 wks post-STZ as Smith et al. (2001, supra) had shown a complete loss of morphine efficacy at 12 wks post-STZ. The dose-normalised %MPE AUC values were significantly (p<0.01) decreased at 3, 9, 12 and 24 wks post-STZ relative to the value determined for protocol control rats, consistent with the findings by Smith et al. (2001, supra).

TABLE 3
Temporal change in the mean (±SEM) area under the degree of
antinociception (% MPE) versus time curve (% MPE A UC values)
following s.c. administration of bolus doses of oxycodone in
control STZ-diabetic rats.

| | | | | Mean | |
|---|---|---|---|---|---|
| | | Mean | (±SEM) dose | | |
| | | (±SEM) | normalised | | |
| Wks | | Oxycod | % MPE | % MPE AUC | |
| Post- | | one dose | AUC | (% MPE | |
| STZ | n | (mg/kg) | (% MPE.h) | AUC.h.kg.mg⁻¹) | Significance |
| Protocol | 6 | 1.2 | 156 (±13.6) | 129.7 (±4.6) | |
| Control | | | | | |
| 3 | 6 | 1.5 | 173 (±4.7) | 115.5 (±3.1) | p < 0.05 |
| 9 | 7 | 2.0 | 162 (±3.2) | 80.8 (±1.6) | n.s. |
| 12 | 6 | 2.0 | 168 (±5.2) | 84.2 (±2.6) | p < 0.01 |
| 24 | 5 | 3.2 | 141 (±7.8) | 44.1 (±2.4) | p < 0.01 |

n.s. = not significant

At 3 wks post-STZ, diabetic rats received an approximate ED₅₀ oxycodone dose (1.5 mg/kg). This was increased to 2.0 mg/kg at 9 and 12 wks post-STZ, indicating a decrease in the antinociceptive potency of oxycodone. There was another dose increase at 24 wks post-STZ to 3.2 mg/kg, consistent with previous studies by Smith et al. (2001, supra). The decreasing values of the dose-normalised %MPE AUC values show that the potency of s.c. oxycodone decreased in a temporal manner control STZ-diabetic rats over the 24 wks post-STZ study period.

TABLE 4
Lack of temporal chance in the mean (±SEM) area under the degree of
antinociception (% MPE) versus time curve (% MPE AUC values)
following s.c. administration of bolus doses of 2.4 mg/kg
morphine in candesartan (2.0 mg/kg/day) treated STZ-diabetic rats.

| | | Morphine | | |
|---|---|---|---|---|
| | | dose | Mean (±SEM) % MPE | |
| Treatment | n | (mg/kg) | AUC (% MPE.h) | Significance |
| Protocol Control | 6 | 2.4 | 149 (±18.8) | |
| 3 wks post-STZ | 6 | 2.4 | 126 (±13.2) | n.s. |
| 9 wks post-STZ | 6 | 2.4 | 138 (±10.9) | n.s. |
| 12 wks post-STZ | 8 | 2.4 | 158 (±10.2) | n.s. |
| 24 wks post-STZ | 8 | 2.4 | 135 (±9.8) | n.s. |

n.s. = not significant

At 3, 9, 12 and 24 wks post-STZ, diabetic rats received the ED₅₀ bolus dose of morphine for control non-diabetic rats previously as determined by Saini, K. (2000, “Differential potency of single-doses of subcutaneous morphine and oxycodone for the relief of mechanical allodynia in Dark Agouti rats with CCI and STZ-diabetic neuropathic pain.” On-Course Hons Research Article, School of Pharmacy, The University of Queensland). The antinociceptive potency of morphine in STZ-diabetic rats that received chronic once-daily administration of oral candesartan (2.0 mg/kg/day) was not significantly different, for the duration of the 24 wk study, to that found in weight-matched control non-diabetic rats that received once-daily oral administration of vehicle (DMSO:water, 10:90).

TABLE 5
Mean (±SEM) area under the degree of antinociception (% MPE)
versus time curve (% MPE AUC values) following administration
of s.c. bolus doses of morphine in candesartan (2.0 mg/kg/day)
treated non-diabetic rats.

| | | Morphine dose | Mean (±SEM) % MPE | |
|---|---|---|---|---|
| Treatment | n | (mg/kg) | AUC (% MPE.h) | Significance |
| Protocol | 6 | 2.4 | 149 (±7.7) | |
| Control | | | | |
| Candesartan | 6 | 2.4 | 140 (±8.3) | n.s. |
| Control | | | | |

n.s. = not significant

High-dose oral candesartan treated (2.0 mg/kg/day) non-diabetic rats received single bolus doses (≈ED₅₀) of morphine (2.4 mg/kg), as previously determined by Saini (2000, supra). Chronic once-daily oral administration of high-dose candesartan did not significantly alter the antinociceptive potency of single bolus doses of s.c. morphine relative to that for non-diabetic protocol control rats.

TABLE 6
Temporal change in the mean
(± SEM) area under the degree of antinociception
(% MPE) versus time curve (% MPE AUC values) following
administration of s.c. bolus doses of oxycodone in high-
dose candesartan (2.0 mg/kg/day) treated STZ-diabetic rats.

| | | Oxycodone | Mean (±SEM) | |
|---|---|---|---|---|
| | | dose | % MPE AUC | |
| Treatment | n | (mg/kg) | (% MPE.h) | Significance |
| Protocol Control | 6 | 1.2 | 156 (±13.6) | |
| 3 wks post-STZ | 6 | 1.2 | 120 (±8.9) | p < 0.05 |
| 9 wks post-STZ | 6 | 1.2 | 136 (±13.3) | n.s. |
| 12 wks post-STZ | 7 | 2.2** | 158 (±6.9) | n.s. |
| 24 wks post-STZ | 6 | 1.2 | 162 (±7.1) | n.s. |

n.s. = not significant

At 3, 9 and 24 wks post-STZ, STZ-diabetic rats received the ED₅₀ dose of s.c oxycodone as previously determined in control non-diabetic rats by Saini (2000, supra). At 3 wks post-STZ, the antinociceptive response (%MPE AUC values) to oxycodone showed a small (≈20%) but significant (p<0.05) decrease in comparison to the non-diabetic protocol control rats. At 9 and 24 wks post-STZ however, the antinociceptive response was not significantly different (p>0.05) from that observed in the non-diabetic protocol control rats, indicating that chronic once-daily administration of anti-hypertensive doses of candesartan (2.0 mg/kg/day) preserved oxycodone potency.

TABLE 7
Mean (±SEM) area under the decree of
antinociception (% MPE) versus time curve (% MPE AUC values)
following administration of bolus doses of s.c. oxycodone in
high-dose oral candesartan (2.0 mg/kg/day) treated non-diabetic rats.

| | | Oxycodone | Mean (±SEM) | |
|---|---|---|---|---|
| | | dose | % MPE AUC | |
| Treatment | n | (mg/kg) | (% MPE.h) | Significance |
| Protocol Control | 6 | 1.2 | 156 (±5.5) | |
| Candesartan Control | 6 | 1.2 | 155 (±8.5) | n.s. |

n.s. = not significant

Candesartan treated (2.0 mg/kg/day) non-diabetic rats received single bolus doses (≈ED₅₀) of oxycodone (1.2 mg/kg), as previously determined by Saini (2000, supra). Chronic once-daily oral administration of high-dose candesartan did not significantly alter the antinociceptive potency of single bolus doses of s.c. oxycodone relative to that for protocol control non-diabetic rats.

TABLE 8
Temporal change in the mean (±SEM) area under the degree of
antinociception (% MPE) versus time curve (% MPE AUC values)
following administration of s.c. bolus doses of moryhine in low-
dose candesartan (0.5 mg/kg/day) treated STZ-diabetic rats.

| | | | | Mean | |
|---|---|---|---|---|---|
| | | | Mean | (±SEM) dose | |
| | | | (±SEM) | normalised | |
| | | Morphine | % MPE | % MPE AUC | |
| | | dose | AUC | (% MPE | Signific- |
| Treatment | n | (mg/kg) | (% MPE.h) | AUC.h.kg.mg⁻¹) | ance |
| Protocol | 6 | 2.4 | 149 (±18.8) | 62.0 (±3.2) | |
| Control | | | | | |
| 3 wks | 6 | 2.4 | 195 (±6.9) | 81.4 (±2.9) | p < 0.01 |
| post-STZ | | | | | |
| 9 wks | 6 | 2.4 | 99 (±12.9) | 41.1 (±5.4) | p < 0.05 |
| post-STZ | | | | | |
| 12 wks | 6 | 14 | 166 (±6.7) | 17.1 (±0.5) | p < 0.01 |
| post-STZ | | | | | |
| 21 wks | 6 | 18 | 71 (±11.2) | 3.9(±0.6) | p < 0.01 |
| post-STZ | | | | | |
| 24 wks | 6 | 18 | 92 (±5.0) | 5.1 (±0.3) | p < 0.01 |
| post-STZ | | | | | |

At 3, 9, 12 and 24 wks post-STZ, approximate ED₅₀ doses for morphine were determined during preliminary dose ranging studies. The significant decrease in the dose-normalised %MPE AUC values shows that there was a significant temporal decrease in morphine potency relative to that in non-diabetic protocol control rats from 9 wks onwards.

TABLE 9
Temporal change in the mean (±SEM) area under the degree of
antinociception (% MPE) versus time curve (% MPE A UC values)
following administration of s.c. bolus doses of oxycodone in low-
dose candesartan (0.5 mg/kg/day) treated STZ-diabetic rats.

| | | | | Mean | |
|---|---|---|---|---|---|
| | | | Mean | (±SEM) dose | |
| | | Oxy- | (±SEM) | normalised | |
| | | codone | % MPE | % MPE AUC | |
| | | Dose | AUC | (% MPE | Signific- |
| Treatment | n | (mg/kg) | (% MPE.h) | AUC.h.kg.mg⁻¹) | ance |
| Protocol | 6 | 1.2 | 149 (±18.8) | 129.7 (±4.593) | |
| Control | | | | | |
| 3 wks | 6 | 1.2 | 131 (±7.9) | 109.5 (±6.549) | n.s. |
| post-STZ | | | | | |
| 9 wks | 6 | 1.2 | 122 (±9.1) | 102.1 (±7.611) | p < 0.05 |
| post-STZ | | | | | |
| 12 wks | 6 | 2.0 | 116 (±8.2) | 67.70 (±4.111) | p < 0.01 |
| post-STZ | | | | | |
| 21 wks | 6 | 2.0 | 171 (±8.3) | 85.34 (±4.134) | p < 0.01 |
| post-STZ | | | | | |
| 24 wk | 6 | 2.0 | 145 (±13.0) | 72.45 (±6.527) | p < 0.01 |
| post-STZ | | | | | |

n.s. = not significant

Bolus s.c. doses (≈ED₅₀) of oxycodone were administered to rats at 3, 9, 12, 21 and 24 wks post-STZ. There was a temporal decrease in the mean (±SEM) value of the dose-normalised %MPE AUC, indicative of a significant decrease in the antinociceptive potency of oxycodone over the 24 wk study period such that by 12 wks post-STZ, there was an approximate 50% decrease in potency in comparison with that observed in protocol controls. This reduction was apparent by 9 wks post-STZ with the same dose (1.2 mg/kg) showing a small (=20%) but significant (p<0.05) decrease in oxycodone potency when compared with non-diabetic protocol control rats.

TABLE 10
Mean (±SEM) area under the degree of antinociception
(% MPE) versus time curve (% MPE AUC values) following
s.c. bolus administration of morphine (2.4 mg/kg) in
STZ-diabetic rats during the “reversal protocol”
pilot study.

| | | | Mean | |
|---|---|---|---|---|
| | | Morphine | (±SEM) % | |
| | | dose | MPE AUC | |
| Treatment | n | (mg/kg) | (% MPE.h) | Significance |
| 24 wks post-STZ with | 6 | 2.4 | 135 (±9.2) | |
| candesartan | | | | |
| (2.0 mg/kg/day) | | | | |
| 6 wks candesartan | 5 | 2.4 | 112 (±14.1) | n.s. |
| cessation | | | | |
| 4 wks candesartan | 4 | 2.4 | 120 (±12.9) | n.s. |
| re-initiation | | | | |
| 6 wks candesartan | 4 | 2.4 | 131 (±7.6) | n.s. |
| re-initiation | | | | |

n.s. = not significant

Cessation of once-daily high-dose oral candesartan (2.0 mg/kg/day) treatment resulted in a small but insignificant decrease in the antinociceptive potency of morphine relative to that observed in the same rats at 24 wks post-STZ but prior to candesartan cessation. Re-initiation of once-daily chronic high-dose oral candesartan (2.0 mg/kg/day) administration completely restored the antinociceptive potency of morphine by six wks of treatment to levels similar to that observed in the same rats prior to the cessation of candesartan treatment.

TABLE 11
Mean (±SEM) area under the antinociception (% MPE)
versus time curve (% MPE AUC values) following s.c.
bolus dose administration of oxvcodone (1.2 mg/kg) in
STZ-diabetic rats during “reversal protocol”
pilot study.

| | | Oxycodone | Mean (±SEM) | |
|---|---|---|---|---|
| | | dose | % MPE AUC | |
| Treatment | n | (mg/kg) | (% MPE.h) | Significance |
| 24 wks post-STZ with | 6 | 1.2 | 162 (±7.1) | |
| candesartan | | | | |
| 6 wks candesartan | 5 | 1.2 | 139 (±11.5) | n.s. |
| cessation | | | | |
| 4 wks candesartan re- | 4 | 1.2 | 157 (±8.5) | n.s. |
| initiation | | | | |
| 6 wks candesartan re- | 4 | 1.2 | 179 (±5.4) | n.s. |
| initiation | | | | |

n.s. = not significant

A small but insignificant decrease in oxycodone potency was apparent by six wks after cessation of candesartan treatment relative to that observed in the same rats at 24 wks post-STZ prior to candesartan (2.0 mg/kg/day) cessation. Re-initiation of chronic once-daily oral candesartan (2.0 mg/kg/day) completely restored the antinociceptive potency of oxycodone levels similar to those observed in the same rats prior to the cessation of candesartan treatment.

What is claimed is:

1. A method for treating or preventing a neuropathic condition in a subject, the method comprising administering to the subject an AT₁ receptor antagonist in an amount that is effective for the treatment or prophylaxis of the neuropathic condition.

2. A method according to claim 1, wherein the neuropathic condition is a primary neuropathic condition.

3. A method according to claim 1, wherein the neuropathic condition is a peripheral neuropathic condition.

4. A method according to claim 1, wherein the neuropathic condition is a painful diabetic neuropathy (PDN).

5. A method according to claim 4, wherein the neuropathic condition is associated with a disorder selected from the group consisting of diabetes, uraemia, amyloidosis, tumaculous neuropathy, nutritional deficiency and kidney failure.

6. A method according to claim 1, wherein the neuropathic condition is selected from the group consisting of hereditary motor and sensory neuropathies (HMSN), hereditary sensory neuropathies (HSNs), hereditary sensory and autonomic neuropathies, and hereditary neuropathies with ulcero-mutilation.

7. A method according to claim 1, wherein the neuropathic condition is associated with a repetitive activity selected from the group consisting of typing and working on an assembly line.

8. A method according to claim 1, wherein the neuropathic condition is associated with trauma.

9. A method according to claim 1, wherein the neuropathic condition is associated with administering to the subject a medication selected from the group consisting of an AIDS medication, an antibiotic, a gold compound, and a chemotherapeutic agent.

10. A method according to claim 9, wherein the medication is selected from the group consisting of nitrofurantoin, dideoxycytosine, dideoxyinosine, metronidazole, vincristine, and cis-platin.

11. A method according to claim 1, wherein the neuropathic condition is associated with exposing the subject to a chemical compound selected from the group consisting of an alcohol, a lead compound, an arsenic compound, a mercury compound, and an organophosphate compound.

12. A method according to claim 1, wherein the condition is associated with an infectious process.

13. A method according to claim 12, wherein the infectious process is selected from the group consisting of Guillian-Barre syndrome HIV and Herpes Zoster (shingles).

14. A method according to claim 1, wherein the AT₁ receptor antagonist is selected from the group consisting of candesartan, eprosartan, irbesartan, losartan, telmisartan, valsartan, tasosartan, olmesartan, E-1477, SC-52458, EXP-3174; BMS-184698, 3-(2′-(tetrazol-5yl)-1,1′-biphenyl-4-yl)methyl-5,7-dimethyl-2-ethyl-3H-imidazo[4,5-b]pyridine and a pharmaceutically compatible salt of any one of these.

15. A method according to claim 14, wherein candesartan is further selected from the group consisting of an analogue of candesartan, a candesartan derivative, a candesartan prodrug and a pharmaceutically compatible salt of any one of these.

16. A method according to claim 1, wherein the subject is normotensive.

17. A method according to claim 1, wherein the AT₁ receptor antagonist is administered to attenuate pain associated with the neuropathic condition.

18. A method according to claim 1, wherein the AT₁ receptor antagonist is administered by a route selected from the group consisting of: injecting parenterally including intramuscular, subcutaneous, intramedullary, intrathecal, intraventricular, intravenous, intraperitoneal, and intraocular routes; applying topically including epithelial, and mucosal delivery such as rectal, vaginal, and intranasal routes; and delivering orally.

19. A method according to claim 1, wherein the AT₁ receptor antagonist is administered orally.

20. A method according to claim 1, wherein the AT₁ receptor antagonist is formulated for sustained release in the subject.

21. A method for preventing or attenuating peripheral neuropathic pain in a subject, the method comprising administering to the subject an amount of candesartan or a pharmaceutically compatible salt thereof that is effective for preventing or attenuating the neuropathic pain.

22. A method for preventing, attenuating or reversing the development of analgesic hyposensitivity to an opioid receptor agonist in a subject, the method comprising administering to the subject an AT₁ receptor antagonist in an amount that is effective for the prevention, attenuation or reversal of the analgesic hyposensitivity to the opioid receptor agonist.

23. A method for producing analgesia in a subject having, or at risk of developing, reduced analgesic sensitivity to an opioid receptor agonist, the method comprising administering to the subject an AT₁ receptor antagonist and an opioid analgesic.

24. A method according to claim 23, wherein the opioid analgesic is the opioid receptor agonist.

25. A method according to claim 23, wherein the AT₁ receptor antagonist is administered in an amount that is effective for reversing the development of analgesic hyposensitivity to the opioid receptor agonist.

26. A method according to claim 23, wherein the AT₁ receptor antagonist is administered in an amount that is effective for reversing the development of tolerance to the opioid receptor agonist.

27. A method according to claim 23, wherein the subject is afflicted with or at risk of developing a neuropathic condition.

28. A method according to claim 27, wherein the neuropathic condition is a peripheral neuropathic condition.

29. A method according to claim 27, wherein the neuropathic condition is PDN.

30. A method according to claim 23, further comprising administering a pharmaceutically acceptable carrier and/or diluent.

31. A method according to claim 23, wherein the opioid analgesic is selected from the group consisting of a μ-opioid receptor agonist, a compound which is metabolised to a μ-opioid receptor agonist and a compound that is converted in vivo to a μ-opioid receptor agonist.

32. A method according to claim 31, wherein the μ-opioid receptor agonist is selected from morphine, methadone, fentanyl, sufentanil, alfentanil, hydromorphone, oxymorphone, their analogues, derivatives or prodrugs and a pharmaceutically compatible salt of any one of these.

33. A method according to claim 31, wherein the μ-opioid receptor agonist is selected from morphine, a morphine analogue, a morphine derivative, a morphine prodrug, and a pharmaceutically compatible salt of any one of these.

34. A method according to claim 23, wherein the opioid analgesic is selected from the group consisting of a κ₂-opioid receptor agonist, a compound which is metabolised to a κ₂-opioid receptor agonist and a compound that is converted in vivo to a κ₂-opioid receptor agonist.

35. A method according to claim 34, wherein the κ₂-opioid receptor agonist is selected from oxycodone, an oxycodone analogue, an oxycodone derivative, an oxycodone prodrug, and a pharmaceutically compatible salt of any one of these.

36. A method according to claim 23, wherein the opioid analgesic is morphine.

37. A method according to claim 23, wherein the opioid analgesic is an oxycodone.

38. A method according to claim 23, wherein the AT₁ receptor antagonist and the opioid analgesic are administered separately.

39. A method according to claim 23, wherein the AT₁ receptor antagonist and the opioid analgesic are administered in a composition in combination.

40. A method according to claim 39, wherein the AT₁ receptor antagonist and the opioid analgesic are administered simultaneously.

41. A method according to claim 23, wherein the subject suffers from reduced opioid analgesic sensitivity.

42. A method according to claim 23, wherein the subject suffers from the development of tolerance to the opioid receptor agonist.

43. A method of preventing or reversing the development of analgesic hyposensitivity to an opioid receptor agonist in a subject, the method comprising administering an AT₁ receptor antagonist together with the opioid receptor agonist.

44. A method of preventing or reversing the development of tolerance to an opioid receptor agonist in a subject, the method comprising administering an AT₁ receptor antagonist and the opioid receptor agonist.

45. A method for producing analgesia in a subject having or at risk of developing a neuropathic condition, the method comprising administering to the subject an AT₁ receptor antagonist in an amount that is effective for preventing, attenuating or reversing a reduced analgesic sensitivity, and an opioid analgesic.

46. A method according to claim 45, wherein the opioid analgesic is an agent to which the subject has reduced analgesic sensitivity.

47. A method according to claim 45, wherein the opioid analgesic is administered in an amount that is effective for the production of analgesia.

48. A method according to claim 45, wherein the condition is a neuropathic condition associated with the development of reduced analgesic sensitivity to an opioid receptor agonist.

49. A method according to claim 48, wherein the opioid analgesic agonises the same opioid receptor as the opioid receptor agonist.

50. An analgesic composition comprising an AT₁ receptor antagonist and an opioid analgesic, each in an amount effective to produce analgesia in a subject having or at risk of developing reduced analgesic sensitivity to an opioid receptor agonist.

51. A composition according to claim 50, wherein the AT₁ receptor antagonist is selected from the group consisting of:
valsartan having the formula: 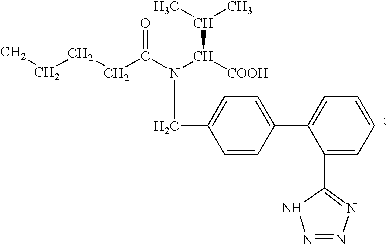
losartan having the following formula: 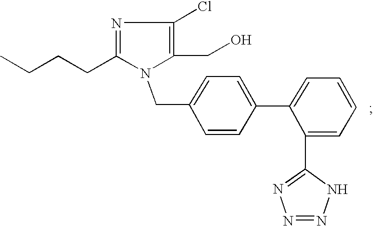
eprosartan having the following formula: 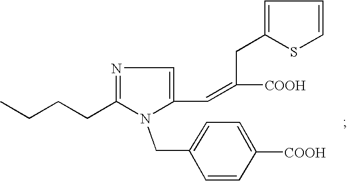
irbesartan having the following formula: 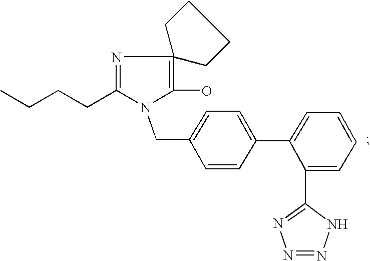
E-1477 having the following formula: 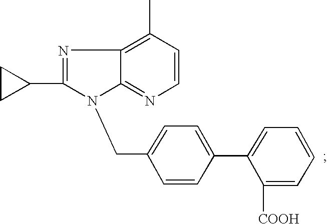
telmisartan having the following formula: 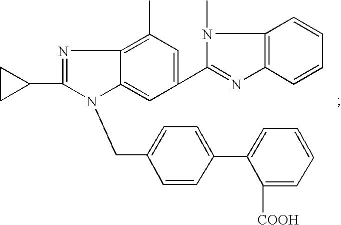
SC-52458 having the following formula: 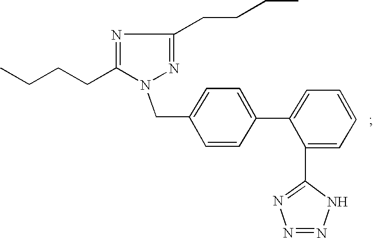
saprisartan having the following formula: 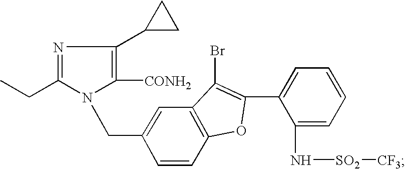
the compound having following formula: 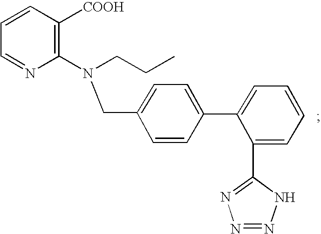
ZD-8731 having the following formula: 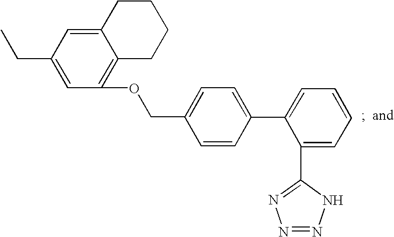
candesartan having the following formula:

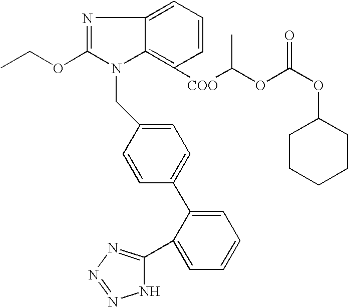

52. A composition according to claim 50, wherein the AT₁ receptor antagonist is selected from the group consisting of tasosartan, olmesartan, EXP-3174; BMS-184698, 3-(2′-(tetrazol-5yl)-1,1′-biphenyl-4-yl)methyl-5,7-dimethyl-2-ethyl-3H-imidazo[4,5-b]pyridine and a pharmaceutically compatible salt of any one of these.

53. A composition according to claim 50, wherein the opioid analgesic agonises the same receptor as the opioid receptor agonist.

54. A composition according to claim 50, wherein the opioid analgesic is the opioid receptor agonist.

55. A composition according to claim 50, wherein the opioid analgesic is selected from the group consisting of a μ-opioid receptor agonist, a compound which is metabolised to a μ-opioid receptor agonist and a compound that is converted in vivo to a μ-opioid receptor agonist.

56. A composition according to claim 55, wherein the μ-opioid receptor agonist is selected from morphine, methadone, fentanyl, sufentanil, alfentanil, hydromorphone, oxyrnorphone, their analogues, derivatives or prodrugs and a pharmaceutically compatible salt of any one of these.

57. A composition according to claim 55, wherein the μ-opioid receptor agonist is selected from morphine, a morphine analogue, a morphine derivative, a morphine prodrug, and a pharmaceutically compatible salt of any one of these.

58. A composition according to claim 50, wherein the opioid analgesic is selected from the group consisting of a κ₂-opioid receptor agonist, a compound which is metabolised to a κ₂-opioid receptor agonist and a compound that is converted in vivo to a κ₂-opioid receptor agonist.

59. A composition according to claim 58, wherein the opioid analgesic is selected from oxycodone, an oxycodone analogue, an oxycodone derivative, an oxycodone prodrug, and a pharmaceutically compatible salt of any one of these.

60. A composition according to claim 50, wherein the opioid analgesic is morphine or oxycodone.

61. A composition according to claim 50, wherein the AT₁ receptor antagonist is candesartan.

62. A composition according to claim 50, further comprising a pharmaceutically acceptable carrier.

63. A composition comprising candesartan and morphine.

64. A composition comprising candesartan and oxycodone.
